(19) United States
(12) Patent Application Publication
(10) Pub. No.: US 2025/0206840 A1
(43) Pub. Date: Jun. 26, 2025

(54) DE-N-ACETYLATED POLYSIALIC ACID (dPSA) BINDING AGENT AND METHOD OF USING SAME

(71) Applicant: Saccharo, Inc., Oakland, CA (US)

(72) Inventors: Gregory Moe, Oakland, CA (US); Leonard G. Presta, South San Francisco, CA (US)

(73) Assignee: Saccharo, Inc., Oakland, CA (US)

(21) Appl. No.: 18/730,282

(22) PCT Filed: Jan. 13, 2023
(86) PCT No.: PCT/US2023/060678

§ 371 Date: Jul. 18, 2024

Related U.S. Application Data
(60) Provisional application No. 63/299,841, filed on Jan. 14, 2022.
(60) Provisional application No. 63/299,843, filed on Jan. 14, 2022.

(51) Int. Cl. C07K 16/30; A61K 39/00; A61K 47/68; A61P 35/00; G01N 33/574

(52) U.S. Cl. CPC C07K 16/30; A61K 47/6851; A61P 35/00; G01N 33/57492; A61K 2039/505; C07K 2317/41; C07K 2317/52; C07K 2317/565; C07K 2317/732; C07K 2317/734; C07K 2317/92

(57) ABSTRACT

A dPSA-binding agent and immunoglobulin heavy chain and light chain polypeptides of the binding agent, as well as methods of using the dPSA-binding agent to treat cancer and kill cancer cells.

### CROSS-REFERENCE TO RELATED APPLICATIONS

This patent application claims the benefit of U.S. Provisional Patent Application 63/299,841, filed Jan. 14, 2022, and U.S. Provisional Patent Application 63/299,843, filed Jan. 14, 2022, which are incorporated by reference in their entireties herein.

### INCORPORATION-BY-REFERENCE OF MATERIAL SUBMITTED ELECTRONICALLY

Incorporated by reference in its entirety herein is a computer-readably nucleotide/amino acid sequence listing submitted concurrently herewith and identified as follows: One 56,304 Byte XMIL file named “514163.xml” created on Jan. 13, 2023.

### BACKGROUND OF THE INVENTION

In general, the goal of anti-cancer immunotherapy has been to identify stable antigens that are highly expressed but not shed or secreted from tumor cells, which antigens can then be used as the basis of immunotherapy, e.g., as the antigen in a cancer vaccine or as a target for antibody-based cancer therapy. Optimally, such tumor antigens are acceptably specific for the cancerous target cells, so as to reduce deleterious side effects that can result from cross-reactivity with non-cancerous cells of the subject being treated. Where cross-reactivity affects cells that can be repopulated, it may be acceptable to relax this requirement for the specificity of immunotherapy.

Altered glycosylation patterns of cell surface proteins occur in nearly all types of cancer. Excessive sialylation of glycoproteins and glycolipids is central to the aberrant regulation of cell adhesion in metastatic cancer, which in turn can result from re-expression and/or overexpression of genes normally expressed during development but not in cells of adult normal tissues. In particular, poly alpha 2->8 N-acetyl neuraminic acid or polylsialic acid (polySia) is expressed mainly during fetal development and is highly restricted to just a few regenerative tissues post development. A de-N-acetylated form of polySia (dPSA) on the surface of cancer but not post-development human cells, and can serve as a tumor antigen for cancer identification and therapy.

Thus, new agents capable of binding cells expressing dPSA are needed.

### BRIEF SUMMARY OF THE INVENTION

Provided herein is a dPSA-binding agent comprising immunoglobulin heavy and light chain polypeptides. Also provided herein is a method of using the dPSA-binding agents to treat cancer, kill cancer cells, and delivering a payload to a cell that expresses dPSA.

Related compositions and methods also are provided, as will be apparent from the following detailed description.

### BRIEF DESCRIPTION OF THE SEVERAL VIEWS OF THE DRAWING(S)

FIG. 1 is a graph depicting mean fluorescence intensity (MFI) of SAC-1 antibody vs. concentration of SEAM 3 reference antibody demonstrating that the test antibody does not bind the same epitope as the reference antibody.

FIG. 2 is a graph depicting mean fluorescence intensity (MFI) of SAC-2 antibody vs. concentration of SEAM 3 reference antibody demonstrating that the test antibody does not bind the same epitope as the reference antibody.

FIGS. 3A and 3B show proteins co-immunoprecipitated by SAC-1, SAC-2, and a control IgG1 antibody on SDS-PAGE.

FIGS. 4A, 4B, and 4C show SAC-3 staining of normal human breast tissue (FIG. 4A) and a breast tumor (FIG. 4C) compared with staining of the tumor by a control IgG2a antibody (FIG. 4B).

FIGS. 5A, 5B, and 5C show graphs depiciting the amount of SAC-1 (FIG. 5A) or SAC-2 antibody (FIG. 5B) vs. relative luminescence units (RLU) demonstrating ADCC for each antibody against various cell lines, as well as quantitative values (FIG. 5C).

FIGS. 6A and 6B are graphs plotting cell kill against concentration of afucosylated SAC-2.1C and SAC-2.1D (referred to as “SAC-2.1CaFUC” and SAC-2.1DaFUC,” respectively) illustrating the effect of afucosylated antibodies on ADCC activity against human A375 melanoma (FIG. 6A) and MDA-MB-231 breast cancer (FIG. 6B) cell lines.

FIGS. 7A and 7B shows the dose-dependence of SAC-1.1 (FIG. 7A) and SAC-2 (FIG. 7B) treatment on tumor growth in a A375 xenograft mouse model of human melanoma.

FIGS. 8A and 8B shows the effect on tumor growth of SAC-1.1, mouse SAC-2, and SAC-2.1C compared to a vehicle control and cyclophosphamide treatment in a MDA-MB-231 xenograft mouse model of human breast cancer (FIG. 8A) and the effect of adding human PBMCs in combination with SAC-2.1C on tumor growth (FIG. 8B).

### DETAILED DESCRIPTION OF THE INVENTION

dPSA is a de-N-acetylated form of polySia (dPSA) found on the surface of cancer cells but not other non-cancerous post development human cells (Granoff et al., J. Immunol. 160(1):5028-36 (1998); Moe et al., J. Immunol. 182(10):6610-7 (2009); Moe et al., Infect Immunol. 73(4): 2123-8 (2005); Moe et al., J. Exp. Clin. Cancer Res. 40(1): 293 (2021); Steirer et al., PLoS ONE 6:e27249 (2011)). Since cell surface dPSA is unique to cancer cells and widely expressed among different cancers, agents that preferentially bind dPSA expressing cells can be used to target cancer cells both for diagnosis and therapy.

Humans have two genes, ST8SIA2 and ST8SIA4, that code for enzymes that synthesize polySia (polysialyltransferases ST8SIA2 and ST8SIA4, respectively). While both genes are highly expressed in humans during fetal development (Angata et al., J. Biol. Chem., 272(11): 7182-90 (1997)), ST8SIA4 is expressed mainly in lymphoid tissues and lymphocytes (Drake et al., PNAS 106(29):11995-2000 (2009)); ST8SIA2 does not appear to be present at significant levels in any adult normal tissues based on Northern blot (Angata et al., J. Biol. Chem., 272(11): 7182-90 (1997)).

Several proteins have been confirmed to be polysialylated in humans (see, e.g., Curreli et al., J. Biol. Chem., 282(42): 30346-56 (2007); Finne et al., Biochem. Biophys. Res. Commun. 112(2): 482-7 (1983); Simon et al., J. Biol. Chem., 288(26):18825-33 (2013); Werneburg et al., Glia, 64(8):1314-30 (2016); Werneburg et al., Glia, 63(7):1240-55 (2015); Yabe et al., J. Biol. Chem., 278(16): 13875-80 (2003)). Neural cell adhesion molecule (NCAM) is the most abundant, particularly during fetal development, and is the most thoroughly investigated (Rutishauser, U., Nat'l Rev. Neurosci., 9(1): 26-35 (2008)). A number of human cancers are reported to express polySia-NCAM abnormally (Amoureaux et al., BMC Cancer, 10: 91 (2010); Gluer et al., Pediatr. Res. 43(1): 145-7 (1998); Roth et al., Am. J. Pathol. 133(2): 227-40 (1988); Tanaka et al., Cancer Res. 60(11): 3072-80 (2000)) where its role in mediating interactions among cells and between cells and the extracellular matrix is associated with metastasis and poor clinical prognosis (Amoureaux et al., BMC Cancer, 10: 91 (2010); Tanaka et al., Cancer Res. 60(11): 3072-80 (2000)). Recently, the inventor identified nucleolin as the protein modified or associated with dPSA, and showed that cell surface dPSA depended on ST8SIA2 expression (Moe et al., J. Exp. Clin. Cancer Res. 40(1): 293 (2021)).

Provided herein is a binding agent (e.g., antibody or antibody fragment) that selectively binds to cells, particularly cancer cells, that express dPSA. In some embodiments, the dPSA binding agent binds nucleolin modified with dPSA Without wishing to be bound by any particular theory or mechanism of action, the binding agents are believed to bind an antigen (e.g., nucleolin) comprising an epitope defined at least in part by one or more dPSA residues. Accordingly, the binding agents are hereinafter referred to as “dPSA binding agents.”

The dPSA-binding agent provided herein comprises Ig heavy chain and light chain polypeptides, each of which comprise at least an Ig heavy chain variable region and an Ig light chain variable region, respectively. The Ig heavy and light chain variable regions, in turn, each comprise three complementarity determining regions (CDRs), usually referred to as CDR1, CDR2, or CDR3. The CDR regions also can be referred to using an “H” or “L” in the nomenclature to denote the heavy or light chain, respectively, i.e., CDRH1, CDRH2, CDRH3, CDRL1, CDRL2, or CDRL3. The CDRs of a given Ig sequence can be determined by any of several conventional numbering schemes, such as Kabat, Chothia, Martin (Enhanced Chothia), IGMT, or AHo (these are commonly used names for numbering schemes widely known in the field and described in published literature see, e.g., Kabat, et al., Sequences of Proteins of Immunological Interest, U.S. Department of Health and Human Services, NIH (1991) describing the “Kabat” numbering scheme; Chothia, et al., Canonical Structures for the Hypervariable Regions of Immunoglobulins, J. Mol. Biol., 196:901-917 (1987) and Al-Lazikani et al., Standard Conformations for the Canonical Structures of Immunoglobulins, J. Mol. Biol., 273:927-948 (1997) describing the “Chothia” numbering scheme, Abhinandan et al., Analysis and Improvements to Kabat and Structurally Correct Numbering of Antibody Variable Domains, Mol. Immunol., 45: 3832-3839 (2008) describing the “Martin” or “Enhanced Chothia” numbering scheme; Lefranc et al., The IMGT unique numberingfor immunoglobulins, T cell Receptors and Ig-like domains, The Immunologist, 7: 132-136 (1999) and Lefranc et al., IMGT unique numbering for immunoglobulin and T cell receptor variable domains and I superfamily V-like domains, Dev. Comp. Immunol., 27: 55-77 (2003) describing the “IMGT” numbering scheme; and Honegger et al., Yet another numbering scheme for immunoglobulin variable domains: an automatic modeling and analysis tool, J. Mol. Biol. 309: 657-670 (2001) describing the “AHo” numbering scheme). The identification of CDRs also can be made through relevant empirical binding data, such as the crystallography studies of the binding agent interactions with its target (e.g., antigen or portion thereof comprising the binding epitope), optionally in conjuction with any of the foregoing numbering systems.

The dPSA-binding agents provided herein are man-made and non-naturally occurring. They have been generated by laboratory techniques and, thus, are properly considered recombinant or synthetic molecules comprising recombinant or synthetic amino acid sequences. The Ig heavy and light chain polypeptides can be “isolated” in the sense that they are removed from the environment in which they are produced (e.g., cell culture) and purified to any degree.

In some embodiments, the dPSA-binding agent comprises an Ig heavy chain variable region comprising any of SEQ ID NOs: 1-4 or at least the CDRs thereof; and an Ig light chain variable region comprising SEQ ID NO: 5 or at least the CDRs thereof. The CDRs can be as determined using any known numbering scheme, such as Kabat, Chothia, Martin (Enhanced Chothia), IGMT, or AHo. In some embodiments, the antibody comprises a heavy chain variable region of any of SEQ ID NOs: 1-4 and light chain variable region of SEQ ID NO: 5, or at least the CDRs thereof as determined by Kabat. In some embodiments, the antibody comprises a heavy chain variable region of any of SEQ ID NOs: 1-4 and light chain variable region of SEQ ID NO: 5, or at least the CDRs thereof as determined by Chothia. In some embodiments, the antibody comprises a heavy chain variable region of any of SEQ ID NOs: 1-4 and light chain variable region of SEQ ID NO: 5, or at least the CDRs thereof as determined by Martin. In some embodiments, the antibody comprises a heavy chain variable region of any of SEQ ID NOs: 1-4 and light chain variable region of SEQ ID NO: 5, or at least the CDRs thereof as determined by IGMT. In some embodiments, the antibody comprises a heavy chain variable region of any of SEQ ID NOs: 1-4 and light chain variable region of SEQ ID NO: 5, or at least the CDRs thereof as determined by AHo. In some embodiments, the dPSA binding agent comprises one of the following combination of Ig heavy and light chain variable regions, or at least the CDRs thereof as determined by any of Kabat, Chothia, Martin (Enhanced Chothia), IGMT, or AHo:

| | Heavy Chain Variable Region | Light Chain Variable Region |
|---|---|---|
| 1 | SEQ ID NO: 1 or CDRs thereof | SEQ ID NO: 5 or CDRs thereof |
| 2 | SEQ ID NO: 2 or CDRs thereof | SEQ ID NO: 5 or CDRs thereof |
| 3 | SEQ ID NO: 3 or CDRs thereof | SEQ ID NO: 5 or CDRs thereof |
| 4 | SEQ ID NO: 4 or CDRs thereof | SEQ ID NO: 5 or CDRs thereof |

In some embodiments, the dPSA-binding agent comprises an Ig heavy chain variable region comprising any of SEQ ID NOs: 17-20 or at least the CDRs thereof; and an Ig light chain variable region comprising SEQ ID NO: 21 or at least the CDRs thereof. The CDRs can be as determined using any known numbering scheme, such as Kabat, Chothia, Martin (Enhanced Chothia), IGMT, or AHo. In some embodiments, the antibody comprises a heavy chain variable region of any of SEQ ID NOs: 17-20 and light chain variable region of SEQ ID NO: 21, or at least the CDRs thereof as determined by Kabat. In some embodiments, the antibody comprises a heavy chain variable region of any of SEQ ID NOs: 17-20 and light chain variable region of SEQ ID NO: 21, or at least the CDRs thereof as determined by Chothia. In some embodiments, the antibody comprises a heavy chain variable region of any of SEQ ID NOs: 17-20 and light chain variable region of SEQ ID NO: 21, or at least the CDRs thereof as determined by Martin. In some embodiments, the antibody comprises a heavy chain variable region of any of SEQ ID NOs: 17-20 and light chain variable region of SEQ ID NO: 21, or at least the CDRs thereof as determined by IGMT. In some embodiments, the antibody comprises a heavy chain variable region of any of SEQ ID NOs: 17-20 and light chain variable region of SEQ ID NO: 21, or at least the CDRs thereof as determined by AHo. In some embodiments, the dPSA binding agent comprises one of the following combination of Ig heavy and light chain variable regions, or at least the CDRs thereof as determined by any of Kabat, Chothia, Martin (Enhanced Chothia), IGMT or AHo:

| | Heavy Chain Variable Region | Light Chain Variable Region |
|---|---|---|
| 1 | SEQ ID NO: 17 or CDRs thereof | SEQ ID NO: 21 or CDRs thereof |
| 2 | SEQ ID NO: 18 or CDRs thereof | SEQ ID NO: 21 or CDRs thereof |
| 3 | SEQ ID NO: 19 or CDRs thereof | SEQ ID NO: 21 or CDRs thereof |
| 4 | SEQ ID NO: 20 or CDRs thereof | SEQ ID NO: 21 or CDRs thereof |

In some embodiments, provided herein is a dPSA binding agent comprising an Ig heavy chain variable region and an Ig light chain variable region, wherein the Ig heavy chain variable region comprises: a CDR1 comprising any one of SEQ ID NOs: 6-9 or 24-27, CDR2 comprising SEQ ID NO: 10 or 28, and CDR3 comprising SEQ ID NO: 11 or 29; and the Ig light chain variable region comprises a CDR1 comprising SEQ ID NO: 12 or 30, CDR2 comprising SEQ ID NO: 13 (RMS) or 31, and CDR3 comprising SEQ ID NO: 14 or 32.

In some embodiments, provided herein is a dPSA binding agent comprising an Ig heavy chain variable region and an Ig light chain variable region, wherein the Ig heavy chain variable region comprises: a CDR1 comprising any one of SEQ ID NOs: 6-9, CDR2 comprising SEQ ID NO: 10, and CDR3 comprising SEQ ID NO: 11; and the Ig light chain variable region comprises a CDR1 comprising SEQ ID NO: 12, CDR2 comprising SEQ ID NO: 13, and CDR3 comprising SEQ ID NO: 14.

| | CDRH1 | CDRH2 | CDRH3 | CDRL1 | CDRL2 | CDRL3 |
|---|---|---|---|---|---|---|
| 1 | SEQ ID | SEQ ID | SEQ ID | SEQ ID | SEQ ID | SEQ ID |
| | NO: 6 | NO: 10 | NO: 11 | NO: 12 | NO: 13 | NO: 14 |
| 2 | SEQ ID | SEQ ID | SEQ ID | SEQ ID | SEQ ID | SEQ ID |
| | NO: 7 | NO: 10 | NO: 11 | NO: 12 | NO: 13 | NO: 14 |
| 3 | SEQ ID | SEQ ID | SEQ ID | SEQ ID | SEQ ID | SEQ ID |
| | NO: 8 | NO: 10 | NO: 11 | NO: 12 | NO: 13 | NO: 14 |
| 4 | SEQ ID | SEQ ID | SEQ ID | SEQ ID | SEQ ID | SEQ ID |
| | NO: 9 | NO: 10 | NO: 11 | NO: 12 | NO: 13 | NO: 14 |

In some embodiments, provided herein is a dPSA binding agent comprising an Ig heavy chain variable region and an Ig light chain variable region, wherein the Ig heavy chain variable region comprises: a CDR1 comprising any one of SEQ ID NOs: 24-27, CDR2 comprising SEQ ID NO: 28, and CDR3 comprising SEQ ID NO: 29; and the Ig light chain variable region comprises a CDR1 comprising SEQ ID NO: 30, CDR2 comprising SEQ ID NO: 31, and CDR3 comprising SEQ ID NO: 32.

| | CDRH1 | CDRH2 | CDRH3 | CDRL1 | CDRL2 | CDRL3 |
|---|---|---|---|---|---|---|
| 1 | SEQ ID NO: | SEQ ID NO: | SEQ ID NO: | SEQ ID NO: | SEQ ID NO: | SEQ ID NO: |
| | 24 | 28 | 29 | 30 | 31 | 32 |
| 2 | SEQ ID NO: | SEQ ID NO: | SEQ ID NO: | SEQ ID NO: | SEQ ID NO: | SEQ ID NO: |
| | 25 | 28 | 29 | 30 | 31 | 32 |
| 3 | SEQ ID NO: | SEQ ID NO: | SEQ ID NO: | SEQ ID NO: | SEQ ID NO: | SEQ ID NO: |
| | 26 | 28 | 29 | 30 | 31 | 32 |
| 4 | SEQ ID NO: | SEQ ID NO: | SEQ ID NO: | SEQ ID NO: | SEQ ID NO: | SEQ ID NO: |
| | 27 | 28 | 29 | 30 | 31 | 32 |
| 5 | SEQ ID NO: | SEQ ID NO: | SEQ ID NO: | SEQ ID NO: | SEQ ID NO: | SEQ ID NO: |
| | 24 | 10 | 11 | 12 | 13 | 14 |
| 6 | SEQ ID NO: | SEQ ID NO: | SEQ ID NO: | SEQ ID NO: | SEQ ID NO: | SEQ ID NO: |
| | 25 | 10 | 11 | 12 | 13 | 14 |
| 7 | SEQ ID NO: | SEQ ID NO: | SEQ ID NO: | SEQ ID NO: | SEQ ID NO: | SEQ ID NO: |
| | 26 | 10 | 11 | 12 | 13 | 14 |
| 8 | SEQ ID NO: | SEQ ID NO: | SEQ ID NO: | SEQ ID NO: | SEQ ID NO: | SEQ ID NO: |
| | 27 | 10 | 11 | 12 | 13 | 14 |

According to yet another aspect of the disclosure, the dPSA-binding agent comprises an Ig heavy chain variable region comprising an amino acid sequence with at least 80% sequence identity (e.g., at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%) to any of SEQ ID NOs: 1-4; and an Ig light chain variable region comprising an amino acid sequence with at least 80% sequence identity (e.g., at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%) to SEQ ID NO: 5. In some embodiments, the dPSA-binding agent comprises an Ig heavy chain polypeptide comprising an amino acid sequence with at least 80% sequence identity (e.g., at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%) to SEQ ID NO: 15; and an Ig light chain polypeptide comprising an amino acid sequence with at least 80% sequence identity (e.g., at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%) to SEQ ID NO: 16. In any of the foregoing embodiments, the Ig heavy chain variable region and Ig light chain variable region can comprise (retain) the CDRs of the heavy and light chain variable regions of sequences, which CDRs can be determined using any known numbering scheme, such as Kabat, Chothia, Martin (Enhanced Chothia), IGMT, or AHo, or as otherwise set forth herein (e.g., SEQ ID NOs: 6-14 above). In some embodiments, the Ig heavy chain variable region comprises one of SEQ ID NOs: 1-4 and the Ig light chain variable region comprises SEQ ID NO: 5. In some embodiments, the dPSA binding agent comprises Ig heavy and light chain polypeptides comprising SEQ ID NO: 15 and SEQ ID NO: 16, respectively.

According to still another aspect of the disclosure, the dPSA-binding agent comprises an Ig heavy chain variable region comprising an amino acid sequence with at least 80% sequence identity (e.g., at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%) to any of SEQ ID NOs: 17-20; and an Ig light chain variable region comprising an amino acid sequence with at least 80% sequence identity (e.g., at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%) to SEQ ID NO: 21. In some embodiments, the dPSA-binding agent comprises an Ig heavy chain polypeptide comprising an amino acid sequence with at least 80% sequence identity (e.g., at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%) to SEQ ID NO: 22; and an Ig light chain polypeptide comprising an amino acid sequence with at least 80% sequence identity (e.g., at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%) to SEQ ID NO: 23. In any of the foregoing embodiments, the Ig heavy chain variable region and Ig light chain variable region can comprise (retain) the CDRs of the heavy and light chain variable regions, which CDRs can be determined using any known numbering scheme, such as Kabat, Chothia, Martin (Enhanced Chothia), IGMT, or AHo, or as otherwise set forth herein (e.g., SEQ ID NOs: 6-14 or 24-32 above). In some embodiments, the Ig heavy and light chain variable regions comprises one of SEQ ID NOs: 17-20 and the Ig light chain variable region comprises SEQ ID NO: 21, respectively. In some embodiments, the dPSA binding agent comprises Ig heavy and light chain polypeptides comprising SEQ ID NO: 22 and SEQ ID NO: 23, respectively.

In some embodiments, the dPSA binding agent comprises Ig heavy and light chain polypeptides comprising SEQ ID NOs: 33 and 34, respectively.

In some embodiments, the dPSA-binding agent comprises an Ig heavy chain variable region comprising SEQ ID NO: 35 or at least the CDRs thereof; and an Ig light chain variable region comprising SEQ ID NO: 36 or at least the CDRs thereof. The CDRs can be as determined using any known numbering scheme, such as Kabat, Chothia, Martin (Enhanced Chothia), IGMT, or AHo. In some embodiments, the antibody comprises a heavy chain variable region of SEQ ID NO: 35 and light chain variable region of SEQ ID NO: 36, or at least the CDRs thereof as determined by Kabat. In some embodiments, the antibody comprises a heavy chain variable region of SEQ ID NO: 35 and light chain variable region of SEQ ID NO: 36, or at least the CDRs thereof as determined by Chothia. In some embodiments, the antibody comprises a heavy chain variable region of SEQ ID NO: 35 and light chain variable region of SEQ ID NO: 36, or at least the CDRs thereof as determined by Martin. In some embodiments, the antibody comprises a heavy chain variable region of SEQ ID NO: 35 and light chain variable region of SEQ ID NO: 36, or at least the CDRs thereof as determined by IGMT. In some embodiments, the antibody comprises a heavy chain variable region of SEQ ID NO: 35 and light chain variable region of SEQ ID NO: 36, or at least the CDRs thereof as determined by AHo.

In some embodiments, the dPSA-binding agent comprises an Ig heavy chain variable region comprising SEQ ID NO: 51 or at least the CDRs thereof; and an Ig light chain variable region comprising SEQ ID NO: 52 or 53 or at least the CDRs thereof. The CDRs can be as determined using any known numbering scheme, such as Kabat, Chothia, Martin (Enhanced Chothia), IGMT, or AHo. In some embodiments, the antibody comprises a heavy chain variable region of SEQ ID NO: 51 and light chain variable region of SEQ ID NO: 52 or 53, or at least the CDRs thereof as determined by Kabat. In some embodiments, the antibody comprises a heavy chain variable region of SEQ ID NO: 51 and light chain variable region of SEQ ID NO: 52 or 53, or at least the CDRs thereof as determined by Chothia. In some embodiments, the antibody comprises a heavy chain variable region of SEQ ID NO: 51 and light chain variable region of SEQ ID NO: 52 or 53, or at least the CDRs thereof as determined by Martin. In some embodiments, the antibody comprises a heavy chain variable region of SEQ ID NO: 51 and light chain variable region of SEQ ID NO: 52 or 53, or at least the CDRs thereof as determined by IGMT. In some embodiments, the antibody comprises a heavy chain variable region of SEQ ID NO: 51 and light chain variable region of SEQ ID NO: 52 or 53, or at least the CDRs thereof as determined by AHo.

In some embodiments, provided herein is a dPSA binding agent comprising an Ig heavy chain variable region and an Ig light chain variable region, wherein the Ig heavy chain variable region comprises: a CDR1 comprising SEQ ID NO: 39 or 45, CDR2 comprising SEQ ID NO: 40 or 46, and CDR3 comprising SEQ ID NO: 41 or 47; and the Ig light chain variable region comprises a CDR1 comprising SEQ ID NO: 42 or 48, CDR2 comprising SEQ ID NO: 43 (GTN), 49, or 56, and CDR3 comprising SEQ ID NO: 44 or 50. In some embodiments, the dPSA binding agent comprises a CDR1 comprising SEQ ID NO: 39, CDR2 comprising SEQ ID NO: 40, and CDR3 comprising SEQ ID NO: 41 or 47; and the Ig light chain variable region comprises a CDR1 comprising SEQ ID NO: 42, CDR2 comprising SEQ ID NO: 43 or 56, and CDR3 comprising SEQ ID NO: 44.

In some embodiments, provided herein is a dPSA binding agent comprising an Ig heavy chain variable region and an Ig light chain variable region, wherein the Ig heavy chain variable region comprises: a CDR1 comprising SEQ ID NO: 45, CDR2 comprising SEQ ID NO: 46, and CDR3 comprising SEQ ID NO: 41 or 47; and the Ig light chain variable region comprises a CDR1 comprising SEQ ID NO: 48, CDR2 comprising SEQ ID NO: 49 or 56, and CDR3 comprising SEQ ID NO: 50.

| | CDRH1 | CDRH2 | CDRH3 | CDRL1 | CDRL2 | CDRL3 |
|---|---|---|---|---|---|---|
| 1 | SEQ ID | SEQ ID | SEQ ID NO: | SEQ ID | SEQ ID NO: | SEQ ID |
| | NO: 39 | NO: 40 | 41 or 47 | NO: 42 | 43 or 56 | NO: 44 |
| 2 | SEQ ID | SEQ ID | SEQ ID | SEQ ID | SEQ ID | SEQ ID |
| | NO: 39 | NO: 40 | NO: 47 | NO: 42 | NO: 43 | NO: 44 |
| 3 | SEQ ID | SEQ ID | SEQ ID | SEQ ID | SEQ ID | SEQ ID |
| | NO: 39 | NO: 40 | NO: 41 | NO: 42 | NO: 43 | NO: 44 |
| 4 | SEQ ID | SEQ ID | SEQ ID | SEQ ID | SEQ ID | SEQ ID |
| | NO: 39 | NO: 40 | NO: 47 | NO: 42 | NO: 56 | NO: 44 |
| 5 | SEQ ID | SEQ ID | SEQ ID | SEQ ID | SEQ ID | SEQ ID |
| | NO: 39 | NO: 40 | NO: 41 | NO: 42 | NO: 56 | NO: 44 |
| 6 | SEQ ID | SEQ ID | SEQ ID NO: | SEQ ID | SEQ ID NO: | SEQ ID |
| | NO: 45 | NO: 46 | 41 or 47 | NO: 48 | 49 or 56 | NO: 50 |
| 7 | SEQ ID | SEQ ID | SEQ ID | SEQ ID | SEQ ID | SEQ ID |
| | NO: 45 | NO: 46 | NO: 47 | NO: 48 | NO: 49 | NO: 50 |
| 8 | SEQ ID | SEQ ID | SEQ ID | SEQ ID | SEQ ID | SEQ ID |
| | NO: 45 | NO: 46 | NO: 41 | NO: 48 | NO: 49 | NO: 50 |
| 9 | SEQ ID | SEQ ID | SEQ ID | SEQ ID | SEQ ID | SEQ ID |
| | NO: 45 | NO: 46 | NO: 47 | NO: 48 | NO: 56 | NO: 50 |
| 10 | SEQ ID | SEQ ID | SEQ ID | SEQ ID | SEQ ID | SEQ ID |
| | NO: 45 | NO: 46 | NO: 41 | NO: 48 | NO: 56 | NO: 50 |

In some embodiments, the dPSA-binding agent comprises an Ig heavy chain variable region comprising an amino acid sequence with at least 80% sequence identity (e.g., at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%) to SEQ ID NO: 35; and an Ig light chain variable region comprising an amino acid sequence with at least 80% sequence identity (e.g., at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%) to SEQ ID NO: 36. In some embodiments, the dPSA-binding agent comprises an Ig heavy chain polypeptide comprising an amino acid sequence with at least 80% sequence identity (e.g., at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%) to SEQ ID NO: 37; and an Ig light chain polypeptide comprising an amino acid sequence with at least 80% sequence identity (e.g., at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%) to SEQ ID NO: 38. In any of the foregoing embodiments, the Ig heavy chain variable region and Ig light chain variable region can comprise (retain) the CDRs of the heavy and light chain variable regions of SEQ ID NO: 35 and 36, respectively, which CDRs can be determined using any known numbering scheme, such as Kabat, Chothia, Martin (Enhanced Chothia), IGMT, or AHo, or as otherwise set forth herein (e.g., SEQ ID NOs: 39-44). In some embodiments, the Ig heavy and light chain variable regions comprise SEQ ID NO: 35 and SEQ ID NO: 36, respectively. In some embodiments, the dPSA binding agent comprises Ig heavy and light chain polypeptides comprising SEQ ID NO: 37 and SEQ ID NO: 38, respectively.

In some embodiments, the dPSA-binding agent comprises an Ig heavy chain variable region comprising an amino acid sequence with at least 80% sequence identity (e.g., at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%) to SEQ ID NO: 51; and an Ig light chain variable region comprising an amino acid sequence with at least 80% sequence identity (e.g., at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%) to SEQ ID NO: 52 or 53. In some embodiments, the dPSA-binding agent comprises an Ig heavy chain polypeptide comprising an amino acid sequence with at least 80% sequence identity (e.g., at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%) to SEQ ID NO: 51; and an Ig light chain polypeptide comprising an amino acid sequence with at least 80% sequence identity (e.g., at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%) to SEQ ID NO: 52 or 53. In any of the foregoing embodiments, the Ig heavy chain variable region and Ig light chain variable region can comprise (retain) the CDRs of the heavy and light chain variable regions of SEQ ID NO: 51 and 52 or 53, respectively, which CDRs can be determined using any known numbering scheme, such as Kabat, Chothia, Martin (Enhanced Chothia), IGMT, or AHo, or as otherwise set forth herein. In some embodiments, the Ig heavy and light chain variable regions comprise SEQ ID NO: 51 and SEQ ID NO: 52 or 53, respectively.

In some embodiments, the dPSA-binding agent comprises an Ig heavy chain variable region comprising an amino acid sequence with at least 80% sequence identity (e.g., at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%) to SEQ ID NO: 57; and an Ig light chain variable region comprising an amino acid sequence with at least 80% sequence identity (e.g., at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%) to SEQ ID NO: 58 or 59. In some embodiments, the dPSA-binding agent comprises an Ig heavy chain polypeptide comprising an amino acid sequence with at least 80% sequence identity (e.g., at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%) to SEQ ID NO: 57; and an Ig light chain polypeptide comprising an amino acid sequence with at least 80% sequence identity (e.g., at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%) to SEQ ID NO: 58 or 59. In any of the foregoing embodiments, the Ig heavy chain variable region and Ig light chain variable region can comprise (retain) the CDRs of the heavy and light chain variable regions of SEQ ID NO: 57 and 58 or 59, respectively, which CDRs can be determined using any known numbering scheme, such as Kabat, Chothia, Martin (Enhanced Chothia), IGMT, or AHo, or as otherwise set forth herein. In some embodiments, the Ig heavy and light chain variable regions comprise SEQ ID NO: 57 and SEQ ID NO: 58 or 59, respectively.

In some embodiments, the dPSA binding agent comprises an Ig heavy and light chain variable region comprising SEQ ID NO: 33 and 34, or at least the CDRs thereof as determined using any known numbering scheme, such as Kabat, Chothia, Martin (Enhanced Chothia), IGMT, or AHo; and/or having at least 80% sequence identity (e.g., at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%) to SEQ ID NO: 33 or 34, optionally while retaining the CDRs thereof. In some embodiments, the dPSA binding agent comprises an Ig heavy and light chain variable region comprising SEQ ID NO: 54 and 55, or at least the CDRs thereof as determined using any known numbering scheme, such as Kabat, Chothia, Martin (Enhanced Chothia), IGMT, or AHo; and/or having at least 80% sequence identity (e.g., at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%) to SEQ ID NO: 54 or 55, optionally while retaining the CDRs thereof.

Sequence “identity” as used in reference to nucleic acid or amino acid sequences can be determined by comparing a nucleic acid or amino acid sequence of interest to a reference nucleic acid or amino acid sequence. The percent identity is the percentage of nucleotides or amino acid residues that are the same (i.e., that are identical) as between the sequence of interest and the reference sequence when optimally aligned. A number of mathematical algorithms for obtaining the optimal alignment and calculating identity between two or more sequences are known and publically available. Examples of such programs include CLUSTAL-W, T-Coffee, and ALIGN (for alignment of nucleic acid and amino acid sequences), BLAST programs (e.g., BLAST 2.1, BL2SEQ, and later versions thereof operated by the National Center for Biotechnology Information, Bethesda, MD) and FASTA programs (e.g., FASTA3x, FAS™, and SSEARCH) (for sequence alignment and sequence similarity searches). Sequence alignment algorithms also are disclosed in, for example, Altschul et al., J. Molecular Biol., 215(3): 403-410 (1990), Beigert et al., Proc. Natl. Acad. Sci. USA, 106(10): 3770-3775 (2009), Durbin et al., eds., Biological Sequence Analysis: Probalistic Models of Proteins and Nucleic Acids, Cambridge University Press, Cambridge, UK (2009), Soding, Bioinformatics, 21(7): 951-960 (2005), Altschul et al., Nucleic Acids Res., 25(17): 3389-3402 (1997), and Gusfield, Algorithms on Strings, Trees and Sequences, Cambridge University Press, Cambridge UK (1997)).

With respect to sequences having less than 100% identity to the heavy and light chain sequences specifically set forth above, one or more amino acids of the aforementioned immunoglobulin heavy chain polypeptides and/or light chain polypeptides can be replaced or substituted with a different amino acid, and/or one of more amino acids can be deleted from or inserted into the disclosed amino acid sequences, provided the biological activity of the polypeptide (e.g., the ability of the dPSA binding agent to bind dPSA) is substantially retained. The biological activity of a dPSA-binding agent can be measured, for example, by the binding affinity for a particular dPSA epitope and/or cross-reactivity with targets other than dPSA. The aforementioned properties or characteristics can be observed, measured, and/or assessed using standard techniques including, but not limited to, ELISA, competitive ELISA, surface plasmon resonance analysis (BIACORE™), or solution phase competition (KINEXA™), as well as other in vitro or in vivo neutralization assays, binding assays, fluorescence-activated cell binding (FACS), or other suitable assays.

The dPSA-binding agent can be part of a multispecific (e.g., bispecific or “dual reactive”) construct (e.g., a multispecific antibody, such as a bispecific or dual reactive antibody) that binds dPSA and another antigen. Such a construct can comprise immunoglobulin heavy and light chain polypeptides that bind dPSA as described herein in combination with immunoglobulin heavy chains and light chains from an immunoglobulin that binds an antigen other than dPSA.

The dPSA binding agent can be part of a conjugate. For example, the dPSA-binding agent can be a conjugate of (1) an anti-dPSA antibody or fragment thereof, and (2) a secondary protein or non-protein moiety. By way of further illustration, the dPSA-binding agent can comprise an anti-dPSA antibody or fragment thereof conjugated to another peptide, a fluorescent molecule, or a chemotherapeutic (e.g., cytotoxic) agent.

In some embodiments, the dPSA-binding agent can be a “whole” immunoglobulin or an antigen-binding immunoglobulin “fragment.” A “whole” immunoglobulin typically consists of four polypeptides: two heavy (H) chain polypeptides and two light (L) chain polypeptides. Each of the heavy chains contains one N-terminal variable (V_{H}) region and three C-terminal constant (C_{H}1, C_{H}2, and C_{H}3) regions, and each light chain contains one N-terminal variable (V_{L}) region and one C-terminal constant (C_{L}) region. The light chains of antibodies can be assigned to one of two distinct types, either kappa (κ) or lambda (λ), based upon the amino acid sequences of their constant domains. In a typical immunoglobulin, each light chain is linked to a heavy chain by disulfide bonds, and the two heavy chains are linked to each other by disulfide bonds. In this configuration, the light chain variable region is generally aligned with the variable region of the heavy chain, and the light chain constant region is generally aligned with the first constant region of the heavy chain. The remaining constant regions of the heavy chains are generally aligned with each other.

The variable regions or hypervariable regions of each pair of light and heavy chains form the antigen binding site of an antibody. The V_{H} and V_{L} regions have the same general structure, with each region comprising four framework (FW or FR) regions. The term “framework region,” as used herein, refers to the relatively conserved amino acid sequences within the variable region, which are located between the hypervariable or complementary determining regions (CDRs). There are four framework regions in each variable domain, which are designated FR1, FR2, FR3, and FR4. The framework regions form the β sheets that provide the structural framework of the variable region (see, e.g., C. A. Janeway et al. (eds.), Immunobiology, 5th Ed., Garland Publishing, New York, NY (2001)). The framework regions are connected by three complementarity determining regions (CDRs). The three CDRs, known as CDR1, CDR2, and CDR3, form the “hypervariable region” of an antibody, which is generally considered to be responsible for antigen binding.

The term “antibody fragment” and like terms (e.g., “fragment of an antibody,” “antibody fragment,” “functional fragment of an antibody”) are used interchangeably herein to mean one or more fragments or portions of an antibody that retain the ability to specifically bind to an antigen (see, generally, Holliger et al., Nat. Biotech., 23(9): 1126-1129 (2005)). Antibody “fragments,” as used herein and routinely in the art, include not only fragments or pieces of a whole antibody in the literal sense, but also other known engineered antibody-like constructs, which might include linkers or other elements that do not naturally occur in a “whole” antibody. Examples of antibody fragments include, but are not limited to, (i) a Fab fragment, which is a monovalent fragment consisting of the V_{L}, V_{H}, C_{L}, and CH₁ domains, (ii) a F(ab′)₂ fragment, which is a bivalent fragment comprising two Fab fragments linked by a disulfide bridge at the hinge region, (iii) a Fv fragment consisting of the V_{L} and V_{H} domains of a single arm of an antibody, (iv) a Fab′ fragment, which results from breaking the disulfide bridge of an F(ab′)₂ fragment using mild reducing conditions, and (v) a disulfide-stabilized Fv fragment (dsFv). The dPSA-binding agent also can be a single chain antibody fragment. Examples of single chain antibody fragments include, but are not limited to, (i) a single chain Fv (scFv), which is a monovalent molecule consisting of the two domains of the Fv fragment (i.e., V_{L} and V_{H}) joined by a synthetic linker which enables the two domains to be synthesized as a single polypeptide chain (see, e.g., Bird et al., Science, 242: 423-426 (1988); Huston et al., Proc. Natl. Acad. Sci. USA, 85: 5879-5883 (1988); and Osbourn et al., Nat. Biotechnol., 16: 778 (1998)) and (ii) a diabody, which is a dimer of polypeptide chains, wherein each polypeptide chain comprises a V_{H} connected to a V_{L} by a peptide linker that is too short to allow pairing between the V_{H} and V_{L} on the same polypeptide chain, thereby driving the pairing between the complementary domains on different V_{H}-V_{L} polypeptide chains to generate a dimeric molecule having two functional antigen binding sites. Any other antigen-binding antibody-like constructs known in the art that comprise Ig heavy and light chain CDRs or variable regions can also be used and are antibody fragments for the purposes of this disclosure. In some embodiments, the dPSA binding agent is (or is part of) a chimeric antigen receptor.

In some embodiments, the dPSA-binding agent comprises a heavy chain constant region, such as a fragment crystallizable (Fc) region or portion thereof. The Fc region can be of any Ig class/subclass (IgA (IgA1, IgA2), IgD, IgE, IgG (IgG1, IgG2, IgG3 and IgG4), IgM, including variants thereof. In some embodiments, the dPSA-binding agent is a “whole” or “complete” Ig (i.e., an antibody); and in other embodiments, the binding agent is an antibody fragment conjugated or linked to an Fc region. In some embodiments, the dPSA binding agent comprises an IgG Fc region, such as IgG1 or IgG4. For instance, the dPSA binding agent can be an IgG1 or IgG4 antibody. In some embodiments, the dPSA-binding agent comprises an Fc region that can mediate complement dependent cytotoxicity (CDC) or antibody-dependent cytotoxicity (ADCC). In some embodiments, the dPSA-binding agent comprises an Fc region that activates natural killer (NK) cells.

The dPSA-binding agent can be a human or humanized antibody, a non-human antibody, or a chimeric antibody. By “chimeric” is meant an antibody or fragment thereof comprising both human and non-human regions. Preferably, the dPSA-binding agent is a humanized antibody. A “humanized” antibody is a monoclonal antibody comprising a human antibody scaffold and at least one CDR obtained or derived from a non-human antibody. Non-human antibodies include antibodies isolated from any non-human animal, such as, for example, a rodent (e.g., a mouse or rat). A humanized antibody can comprise, one, two, or three CDRs obtained or derived from a non-human antibody.

A human antibody, a non-human antibody, a chimeric antibody, or a humanized antibody can be obtained by any means, including via in vitro sources (e.g., a hybridoma or a cell line producing an antibody recombinantly) and in vivo sources (e.g., rodents). Methods for generating antibodies are known in the art and are described in, for example, Köhler and Milstein, Eur. J. Immunol., 5: 511-519 (1976); Harlow and Lane (eds.), Antibodies: A Laboratory Manual, CSH Press (1988); and Janeway et al. (eds.), Immunobiology, 5th Ed., Garland Publishing, New York, NY (2001)). In certain embodiments, a human antibody or a chimeric antibody can be generated using a transgenic animal (e.g., a mouse) wherein one or more endogenous immunoglobulin genes are replaced with one or more human immunoglobulin genes (see, e.g., Lonberg, Nat. Biotechnol., 23(9): 1117-25 (2005), and Lonberg, Handb. Exp. Pharmacol., 181: 69-97 (2008)). A humanized antibody can be generated using any suitable method known in the art (see, e.g., An, Z. (ed.), Therapeutic Monoclonal Antibodies: From Bench to Clinic, John Wiley & Sons, Inc., Hoboken, New Jersey (2009)), including, e.g., grafting of non-human CDRs onto a human antibody scaffold (see, e.g., Kashmiri et al., Methods, 36(1): 25-34 (2005); and Hou et al., J. Biochem., 144(1): 115-120 (2008)).

The dPSA-binding agent provided herein can be used for any purpose. For example, the dPSA-binding agent can be used for targeting or killing a cancer cell that expresses dPSA (e.g., that comprises dPSA on the cell surface). Thus, provided herein is a method of targeting or killing a cancer cell, in vitro or in vivo, comprising administering to the cancer cell a dPSA binding agent as described herein. When the method is used to target or kill a cancer cell in vivo, the dPSA binding agent can be administered to the cancer cell by administering the dPSA binding agent to the subject comprising the cancer cell.

The cancer cell expresses can be any cancer cell that expresses dPSA on the cell surface. For instance, the cancer cell can comprise a protein such as nucleolin with dPSA linked to or otherwise associated with the protein on the cell surface). In some embodiments, the cancer cell expresses ST8SIA2.

The method of targeting or killing a cancer cell can be used for diagnostic or therapeutic purposes, and can be used in vitro, ex vivo, or in vivo. For instance, the dPSA binding agent can be conjugated to a detectable label or support (e.g., radiolabel, fluorescent label, beads, scaffold, or the like) to analyze dPSA expression levels or facilitate detection of cancer cells expressing dPSA in a biological sample from a subject (biological fluid or tissue sample). For example, the detectable moiety can be a radioisotope (e.g., ³H, ¹⁴C, ³²P, ³⁵S, or ¹²⁵I), a fluorescent or chemiluminescent compound (e.g., fluorescein isothiocyanate, rhodamine, or luciferin), an enzyme (e.g., alkaline phosphatase, beta-galactosidase, or horseradish peroxidase), or a support (bead, scaffold, biosensor surface, etc). Any method known in the art for separately conjugating an antigen-binding agent (e.g., an antibody) to a such moieties may be employed in the context of the invention (see, e.g., Hunter et al., Nature, 194: 495-496 (1962); David et al., Biochemistry, 13: 1014-1021 (1974); Pain et al., J. Immunol. Meth., 40: 219-230 (1981); and Nygren, J. Histochem. and Cytochem., 30: 407-412 (1982)).

Alternatively, the dPSA binding agent can be used to kill cancer cells. For instance, the dPSA binding agent can be conjugated to a chemotherapeutic agent (e.g., a cytotoxic agent) and used to target and deliver the chemotherapeutic agent to the cancer cell in a expressing dPSA, thereby killing the cancer cell in a subject. In other embodiments, the dPSA binding agent exhibits antibody-dependent cell cytotoxicity (ADCC) or complement-dependent cytotoxicity (CDC), and is used to kill a cancer cell expressing dPSA by binding the cancer cell and effecting ADCC or CDC mediated cell death. In some embodiments, the dPSA binding agent is afucosylated. Afucosylated binding agents (e.g., antibodies) can be prepared by any suitable technique, such as by expressing a nucleic acid encoding the Ig heavy and light chains of the dPSA binding agent in a cell line in which the FUT8 gene has been disrupted or deleted (e.g., FUT8-deleted CHO cells).

Thus, the dPSA binding agent provided herein, and methods of using same, can be used to treat cancer characterized by dPSA surface expression. As used herein, the terms “treatment,” “treating,” and the like refer to obtaining a desired pharmacologic and/or physiologic effect, e.g., to reduce the severity, or inhibit the progress, of a disease and/or adverse symptom attributable to the disease. To this end, the inventive method comprises administering a “therapeutically effective amount” of the dPSA-binding agent. A “therapeutically effective amount” refers to an amount effective, at dosages and for periods of time necessary, to achieve a desired therapeutic result. The therapeutically effective amount may vary according to factors such as the disease state, age, sex, and weight of the individual, and the ability of the dPSA binding agent to elicit a desired response in the individual.

The methods and compositions provided herein are useful in the context of treating or preventing a wide variety of cancers, including carcinomas, sarcomas, leukemias, myelomas, and lymphomas.

Carcinomas that can be amenable to therapy by a method disclosed herein include, but are not limited to, esophageal carcinoma, hepatocellular carcinoma, basal cell carcinoma (a form of skin cancer), squamous cell carcinoma (various tissues), bladder carcinoma, including transitional cell carcinoma (a malignant neoplasm of the bladder), bronchogenic carcinoma, colon carcinoma, colorectal carcinoma, gastric carcinoma, lung carcinoma, including small cell carcinoma and non-small cell carcinoma of the lung, adrenocortical carcinoma, thyroid carcinoma, pancreatic carcinoma, breast carcinoma, ovarian carcinoma, prostate carcinoma, adenocarcinoma, sweat gland carcinoma, sebaceous gland carcinoma, papillary carcinoma, papillary adenocarcinoma, cystadenocarcinoma, medullary carcinoma, renal cell carcinoma, ductal carcinoma in situ or bile duct carcinoma, choriocarcinoma, seminoma, embryonal carcinoma, Wilm's tumor, cervical carcinoma, uterine carcinoma, testicular carcinoma, osteogenic carcinoma, epithelial carcinoma, and nasopharyngeal carcinoma.

Sarcomas that can be amenable to therapy by a method disclosed herein include, but are not limited to, fibrosarcoma, myxosarcoma, liposarcoma, chondrosarcoma, chordoma, osteogenic sarcoma, osteosarcoma, angiosarcoma, endotheliosarcoma, lymphangiosarcoma, lymphangioendotheliosarcoma, synovioma, mesothelioma, Ewing's sarcoma, leiomyosarcoma, rhabdomyosarcoma, and other soft tissue sarcomas.

Other solid tumors that can be amenable to therapy by a method disclosed herein include, but are not limited to, glioma, astrocytoma, medulloblastoma, craniopharyngioma, ependymoma, pinealoma, hemangioblastoma, acoustic neuroma, oligodendroglioma, menangioma, melanoma, neuroblastoma, and retinoblastoma.

Other cancers include leuckemias, lymphomas, and myelomas (including multiple myeloma). Leukemias that can be amenable to therapy by a method disclosed herein include, but are not limited to, a) chronic myeloproliferative syndromes (neoplastic disorders of multipotential hematopoietic stem cells); b) acute myelogenous leukemias (neoplastic transformation of a multipotential hematopoietic stem cell or a hematopoietic cell of restricted lineage potential); c) chronic lymphocytic leukemias (CLL; clonal proliferation of immunologically immature and functionally incompetent small lymphocytes), including B-cell CLL, T-cell CLL prolymphocytic leukemia, and hairy cell leukemia; and d) acute lymphoblastic leukemias (characterized by accumulation of lymphoblasts). Lymphomas that can be treated using a subject method include, but are not limited to, B-cell lymphomas (e.g., Burkitt's lymphoma); Hodgkin's lymphoma; non-Hodgkin's lymphoma, and the like.

Other cancers that can be amenable to treatment according to the methods disclosed herein include atypical meningioma (brain), islet cell carcinoma (pancreas), medullary carcinoma (thyroid), mesenchymal (intestine), hepatocellular carcinoma (liver), hepatoblastoma (liver), clear cell carcinoma (kidney), and neurofibroma mediastinum.

Further exemplary cancers that can be amenable to treatment using a methods disclosed herein include, but art not limited to, cancers of neuroectodermal and epithelial origin. Examples of cancers of neuroectodermal origin include, but are not limited to, Ewing's sarcoma, spinal tumors, brain tumors, supratenbrial primitive neuroectodermal tumors of infancy, tubulocystic carcinoma, mucinous tubular and spindle cell carcinoma, renal tumors, mediastinum tumors, neurogliomas, neuroblastomas, and sarcomas in adolescents and young adults. Examples of epithelial origin include, but are not limited to, small cell lung cancer, cancers of the breast, eye lens, colon, pancreas, kidney, liver, ovary, and bronchial epithelium. In some embodiments, the subject methods do not include treatment of melanoma (i.e., the cancer is other than melanoma). In other embodiments, the subject methods do not include treatment of lymphoma (i.e., the cancer is other than lymphoma).

The dPSA-binding agent can be part of a composition suitable for administration to a mammal. Preferably, the composition is a pharmaceutically acceptable (e.g., physiologically acceptable) composition, which comprises a carrier, preferably a pharmaceutically acceptable (e.g., physiologically acceptable) carrier, and the inventive amino acid sequences, antigen-binding agent, or vector. Any suitable carrier can be used within the context of the invention, and such carriers are well known in the art. The choice of carrier will be determined, in part, by the particular site to which the composition may be administered and the particular method used to administer the composition. The composition also can comprise any other excipient used in the formulation of therapeutic molecules (e.g., proteins or antibodies), particularly parenteral formulations, including, for instance, buffers, tonicity modifiers, stabilizers, surfactants and the like. The composition can be sterile. The composition can be frozen or lyophilized for storage and reconstituted in a suitable sterile carrier prior to use. The compositions can be generated in accordance with conventional techniques described in, e.g., Remington: The Science and Practice of Pharmacy, 21st Edition, Lippincott Williams & Wilkins, Philadelphia, PA (2001).

Administration may be effected using any standard administration techniques, including oral, intravenous, intraperitoneal, subcutaneous, pulmonary, transdermal, intramuscular, intranasal, buccal, sublingual, or suppository administration. The composition preferably is suitable for parenteral administration. The term “parenteral,” as used herein, includes intravenous, intramuscular, subcutaneous, rectal, vaginal, and intraperitoneal administration. More preferably, the composition is administered to a mammal using peripheral systemic delivery by intravenous, intraperitoneal, or subcutaneous injection.

The dPSA-binding agent of the invention may be administered alone or in combination with other drugs. For example, the dPSA-binding agent can be administered in combination with other agents for the treatment or prevention of the diseases disclosed herein, such as other anti-cancer agents. In this respect, for example, the dPSA-binding agent can be used in combination with at least one other agent including, for example, chemotherapeutic agents, vaccines, biological therapies (e.g., other monoclonal antibodies), radiation therapy, bone marrow transplantation, chemotherapeutic treatment, biological response modifier treatment and/or surgery.

Further provided herein is a nucleic acid that encodes the dPSA-binding agent (i.e., encodes the immunoglobulin heavy chain polypeptide and/or the immunoglobulin light chain polypeptide of the dPSA binding agent).

The nucleic acid can be a polymer of DNA or RNA (or both, such as hybrid DNA/RNA), which can be single-stranded or double-stranded and which can contain non-natural or altered nucleotides. The nucleic acid can be part of a vector. The vector can be, for example, a plasmid, episome, cosmid, viral vector (e.g., retroviral or adenoviral), or phage. Suitable vectors and methods of vector preparation are well known in the art (see, e.g., Sambrook et al., Molecular Cloning, a Laboratory Manual, 3rd edition, Cold Spring Harbor Press, Cold Spring Harbor, N.Y. (2001), and Ausubel et al., Current Protocols in Molecular Biology, Greene Publishing Associates and John Wiley & Sons, New York, N.Y. (1994)).

The vector typically comprises expression control sequences, such as a promoter, enhancer, polyadenylation signal, transcription terminator, signal peptide (e.g., the osteonectin signal peptide), internal ribosome entry site (IRES), and the like, that provide for the expression of the coding sequence in a host cell. Exemplary expression control sequences are known in the art and described in, for example, Goeddel, Gene Expression Technology: Methods in Enzymology, Vol. 185, Academic Press, San Diego, Calif. (1990).

A large number of promoters, including constitutive, inducible, and repressible promoters, from a variety of different sources are well known in the art. Representative sources of promoters include for example, virus, mammal, insect, plant, yeast, and bacteria, and suitable promoters from these sources are readily available, or can be made synthetically, based on sequences publicly available, for example, from depositories such as the ATCC as well as other commercial or individual sources. Promoters can be unidirectional (i.e., initiate transcription in one direction) or bi-directional (i.e., initiate transcription in either a 3′ or 5′ direction). Non-limiting examples of promoters include, for example, the T7 bacterial expression system, pBAD (araA) bacterial expression system, the cytomegalovirus (CMV) promoter, the SV40 promoter, the RSV promoter. Inducible promoters include, for example, the Tet system (U.S. Pat. Nos. 5,464,758 and 5,814,618), the Ecdysone inducible system (No et al., Proc. Natl. Acad. Sci., 93: 3346-3351 (1996)), the T-REX™ system (Invitrogen, Carlsbad, CA), LACSWITCH™ system (Stratagene, San Diego, CA), and the Cre-ERT tamoxifen inducible recombinase system (Indra et al., Nuc. Acid. Res., 27: 4324-4327 (1999); Nuc. Acid. Res., 28: e99 (2000); U.S. Pat. No. 7,112,715; and Kramer & Fussenegger, Methods Mol. Biol., 308: 123-144 (2005)).

The term “enhancer” as used herein, refers to a DNA sequence that increases transcription of, for example, a nucleic acid sequence to which it is operably linked. Enhancers can be located many kilobases away from the coding region of the nucleic acid sequence and can mediate the binding of regulatory factors, patterns of DNA methylation, or changes in DNA structure. A large number of enhancers from a variety of different sources are well known in the art and are available as or within cloned polynucleotides (from, e.g., depositories such as the ATCC as well as other commercial or individual sources). A number of polynucleotides comprising promoters (such as the commonly-used CMV promoter) also comprise enhancer sequences. Enhancers can be located upstream, within, or downstream of coding sequences.

The vector also can comprise a “selectable marker gene.” The term “selectable marker gene,” as used herein, refers to a nucleic acid sequence that allow cells expressing the nucleic acid sequence to be specifically selected for or against, in the presence of a corresponding selective agent. Suitable selectable marker genes are known in the art and described in, e.g., International Patent Application Publications WO 1992/008796 and WO 1994/028143; Wigler et al., Proc. Natl. Acad. Sci. USA, 77: 3567-3570 (1980); O'Hare et al., Proc. Natl. Acad. Sci. USA, 78: 1527-1531 (1981); Mulligan & Berg, Proc. Natl. Acad. Sci. USA, 78: 2072-2076 (1981); Colberre-Garapin et al., J. Mol. Biol., 150: 1-14 (1981); Santerre et al., Gene, 30: 147-156 (1984); Kent et al., Science, 237: 901-903 (1987); Wigler et al., Cell, 11: 223-232 (1977); Szybalska & Szybalski, Proc. Natl. Acad. Sci. USA, 48: 2026-2034 (1962); Lowy et al., Cell, 22: 817-823 (1980); and U.S. Pat. Nos. 5,122,464 and 5,770,359.

In some embodiments, the vector is an “episomal expression vector” or “episome,” which is able to replicate in a host cell, and persists as an extrachromosomal segment of DNA within the host cell in the presence of appropriate selective pressure (see, e.g., Conese et al., Gene Therapy, 11: 1735-1742 (2004)). Representative commercially available episomal expression vectors include, but are not limited to, episomal plasmids that utilize Epstein Barr Nuclear Antigen 1 (EBNA1) and the Epstein Barr Virus (EBV) origin of replication (oriP). The vectors pREP4, pCEP4, pREP7, and pcDNA3.1 from Invitrogen (Carlsbad, CA) and pBK-CMV from Stratagene (La Jolla, CA) represent non-limiting examples of an episomal vector that uses T-antigen and the SV40 origin of replication in lieu of EBNA1 and oriP.

Other suitable vectors include integrating expression vectors, which may randomly integrate into the host cell's DNA, or may include a recombination site to enable the specific recombination between the expression vector and the host cell's chromosome. Such integrating expression vectors may utilize the endogenous expression control sequences of the host cell's chromosomes to effect expression of the desired protein. Examples of vectors that integrate in a site specific manner include, for example, components of the flp-in system from Invitrogen (Carlsbad, CA) (e.g., pcDNA™5/FRT), or the cre-lox system, such as can be found in the pExchange-6 Core Vectors from Stratagene (La Jolla, CA). Examples of vectors that randomly integrate into host cell chromosomes include, for example, pcDNA3.1 (when introduced in the absence of T-antigen) from Life Technologies (Carlsbad, CA), UCOE from Millipore (Billerica, MA), and pCI or pFN10A (ACT) FLEXI™ from Promega (Madison, WI).

Viral vectors also can be used. Representative commercially available viral expression vectors include, but are not limited to, the adenovirus-based Per.C6 system available from Crucell, Inc. (Leiden, The Netherlands), the lentiviral-based pLP1 from Invitrogen (Carlsbad, CA), and the retroviral vectors pFB-ERV plus pCFB-EGSH from Stratagene (La Jolla, CA).

Nucleic acid sequences encoding the inventive amino acid sequences can be provided to a cell on the same vector (i.e., in cis). A unidirectional promoter can be used to control expression of each nucleic acid sequence. In another embodiment, a combination of bidirectional and unidirectional promoters can be used to control expression of multiple nucleic acid sequences. Nucleic acid sequences encoding the inventive amino acid sequences alternatively can be provided to the population of cells on separate vectors (i.e., in trans). Each of the nucleic acid sequences in each of the separate vectors can comprise the same or different expression control sequences. The separate vectors can be provided to cells simultaneously or sequentially

The vector(s) comprising the nucleic acid(s) encoding the inventive amino acid sequences can be introduced into a host cell that is capable of expressing the polypeptides encoded thereby, including any suitable prokaryotic or eukaryotic cell. As such, the invention provides an isolated cell comprising the inventive vector. Preferred host cells are those that can be easily and reliably grown, have reasonably fast growth rates, have well characterized expression systems, and can be transformed or transfected easily and efficiently.

Examples of suitable prokaryotic cells include, but are not limited to, cells from the genera Bacillus (such as Bacillus subtilis and Bacillus brevis), Escherichia (such as E coli), Pseudomonas, Streptomyces, Salmonella, and Erwinia. Particularly useful prokaryotic cells include the various strains of Escherichia coli (e.g., K12, HB101 (ATCC No. 33694), DH5α, DH10, MC1061 (ATCC No. 53338), and CC102).

In some embodiments, the vector is introduced into a eukaryotic cell. Suitable eukaryotic cells are known in the art and include, for example, yeast cells, insect cells, and mammalian cells. Examples of suitable yeast cells include those from the genera Kluyveromyces, Pichia, Rhino-sporidium, Saccharomyces, and Schizosaccharomyces. Preferred yeast cells include, for example, Saccharomyces cerivisae and Pichia pastoris.

Suitable insect cells are described in, for example, Kitts et al., Biotechniques, 14: 810-817 (1993); Lucklow, Curr. Opin. Biotechnol., 4: 564-572 (1993); and Lucklow et al., J. Virol., 67: 4566-4579 (1993). Preferred insect cells include Sf-9 and HI5 (Invitrogen, Carlsbad, CA).

In some embodiments, mammalian cells are utilized in the invention. A number of suitable mammalian host cells are known in the art, and many are available from the American Type Culture Collection (ATCC, Manassas, VA). Examples of suitable mammalian cells include, but are not limited to, Chinese hamster ovary cells (CHO) (e.g., CHO-K1 cells, ATCC No. CCL61), CHO DHFR-cells (Urlaub et al., Proc. Natl. Acad. Sci. USA, 97: 4216-4220 (1980)), human embryonic kidney (HEK) 293 or 293T cells (ATCC No. CRL1573), and 3T3 cells (ATCC No. CCL92). Other suitable mammalian cell lines are the monkey COS-1 (ATCC No. CRL1650) and COS-7 cell lines (ATCC No. CRL1651), as well as the CV-1 cell line (ATCC No. CCL70). Further exemplary mammalian host cells include primate cell lines and rodent cell lines, including transformed cell lines. Normal diploid cells, cell strains derived from in vitro culture of primary tissue, as well as primary explants, are also suitable. Other suitable mammalian cell lines include, but are not limited to, mouse neuroblastoma N2A cells, HeLa, mouse L-929 cells, and BHK or HaK hamster cell lines, all of which are available from the ATCC. Methods for selecting suitable mammalian host cells and methods for transformation, culture, amplification, screening, and purification of cells are known in the art.

In one embodiment, the mammalian cell is a human cell. For example, the mammalian cell can be a human lymphoid or lymphoid derived cell line, such as a cell line of pre-B lymphocyte origin. Examples of human lymphoid cells lines include, without limitation, RAMOS (CRL-1596), Daudi (CCL-213), EB-3 (CCL-85), DT40 (CRL-2111), 18-81 (Jack et al., Proc. Natl. Acad. Sci. USA, 85: 1581-1585 (1988)), Raji cells (CCL-86), PER.C6 cells (Crucell Holland B. V., Leiden, The Netherlands), and derivatives thereof.

A nucleic acid sequence encoding the inventive amino acid sequence may be introduced into a cell by any suitable method, such as by “transfection,” “transformation,” or “transduction.” “Transfection,” “transformation,” or “transduction,” as used herein, refer to the introduction of one or more exogenous polynucleotides into a host cell by using physical or chemical methods. Many suitable techniques are known in the art and include, for example, calcium phosphate DNA co-precipitation (see, e.g., Murray E. J. (ed.), Methods in Molecular Biology, Vol. 7, Gene Transfer and Expression Protocols, Humana Press (1991)); DEAE-dextran; electroporation; cationic liposome-mediated transfection; tungsten particle-facilitated microparticle bombardment (Johnston, Nature, 346: 776-777 (1990)); and strontium phosphate DNA co-precipitation (Brash et al., Mol. CellBiol., 7: 2031-2034 (1987)). Phage or viral vectors can be introduced into host cells, after growth of infectious particles in suitable packaging cells, many of which are commercially available.

The nucleic acids and cells can be used for any purpose, such as for the manufacture of the dPSA-binding agent described herein. In this respect, the invention provides a method of preparing the dPSA-binding agent comprising culturing a cell comprising a nucleic acid or nucleic acid sequences encoding the heavy and/or light immunoglobulin polypeptides of the dPSA-binding agent. Phrased differently, the method comprises expressing a nucleic acid encoding the immunoglobulin heavy and/or light chains of the dPSA-binding agent in a cell (e.g., an in vitro cell, such as any of the cell lines discussed herein including CHO and CHO-K1 cells). It will be appreciated that the immunoglobulin heavy and light chains can be expressed from a single nucleic acid in a given cell, or the immunoglobulin heavy and light chains can be expressed from separate nucleic acids in the same cell. The method can further comprise harvesting and/or purifying the dPSA-binding agent from the cell or cell culture media using known techniques.

The following examples further illustrate the invention but, of course, should not be construed as in any way limiting its scope.

### Example 1

The following example illustrates the selective binding of dPSA binding agents provided herein to dPSA antigen.

Antibodies designated SAC-1 and SAC-2 having the sequences set forth below were expressed as a recombinant chimera with a human IgG1 Fc in a CHO cell line that does not express dPSA. The antibodies were tested for specific binding to dPSA by ELISA and cell based assay. Humanized antibodies were prepared with the same CDRs as the SAC-1 and SAC-2 antibodies. The antibodies were designated SAC-1.1, SAC-2.1 (also referred to as “SAC-2 humanized D”), and SAC-2.2 (also referred to as “SAC-2 humanized C”), and have sequences as set forth below.

| | Heavy Chain | Light Chain | Full Heavy | Full Light |
|---|---|---|---|---|
| Antibody | Variable Region | Variable Region | Chain | Chain |
| SAC-1 | SEQ ID NO: 1 | SEQ ID NO: 5 | SEQ ID NO: 15 | SEQ ID NO: 16 |
| SAC-2 | SEQ ID NO: 35 | SEQ ID NO: 36 | SEQ ID NO: 37 | SEQ ID NO: 38 |
| SAC-1.1 | SEQ ID NO: 17 | SEQ ID NO: 21 | SEQ ID NO: 22 | SEQ ID NO: 23 |
| SAC-1.1 M1 | SEQ ID NO: 18 | SEQ ID NO: 21 | | |
| SAC-1.1 M2 | SEQ ID NO: 19 | SEQ ID NO: 21 | | |
| SAC-1.1 M3 | SEQ ID NO: 20 | SEQ ID NO: 21 | | |
| SAC-2.1 | SEQ ID NO: 51 | SEQ ID NO: 52 | SEQ ID NO: 57 | SEQ ID NO: 58 |
| SAC-2.2 | SEQ ID NO: 51 | SEQ ID NO: 53 | SEQ ID NO: 57 | SEQ ID NO: 59 |

dPSA Antigen Preparation

dPSA antigen for use in ELISA was prepared by combining 100 milligrams of colominic acid (MilliporeSigma), 10 milligrams of sodium borohydride (MilliporeSigma) in 10 milliliters of 2 molar sodium and heated to 100 degrees C. for 40 minutes The resulting dPSA was neutralized with 2M hydrochloric acid, dialyzed against 4 liters of water 2 times and lyophilized. dPSA (20 milligrams) in 0.75 milliliters of 0.1M sodium acetate, pH 6.5 was first oxidized with sodium periodate (0.25 milliliters of 10 millimolar periodate) for 30 minutes at ambient temperature in the dark. After adding 100 microliters of 10% (volume/volume) ethylene glycol, the reaction mixture was dialyzed and lyophilized as above. 20 milligrams of oxidized dPSA and 10 milligrams of ovalbumin (IMJECT™ Ovalbumin, Pierce Chemical, Company) were combined in phosphate buffered saline (PBS) containing ˜5 milligrams of sodium cyanoborohydride. The solution was stirred overnight at ambient temperature in the dark. The dPSA-ovalbumin conjugate was purified by size exclusion chromatography on a ToyoPearl HW-65F column in 0.9% (weight/volume) sodium chloride, 10 mM potassium phosphate, pH 7.1. Fractions containing the dPSA-KLH conjugate were concentrated (SpinX, Corning) to 2 milligrams per milliliter protein and 4 milligrams per milliliter dPSA containing ˜30% de-N-acetyl residues determined by modified resorcinol assay.

Binding to dPSA Antigen

ELISAs were performed as described in Moe et al., J. Exp. & Clin. Can. Res. 40(1):293 (2021), starting at an antibody concentration of 10 micrograms per ml with eight serial 3-fold dilutions. Specificity for dPSA was determined by performing the same ELISA in the presence of 100 micrograms of polysialic acid (i.e. colominic acid, MilliporeSigma) in the buffer. The results (OD405 of >0.5 Mean Fluorescence Intensity (MFI) in 30 minutes) confirmed that the antibody specifically bound dPSA.

The antibody also was tested for binding to human neuroblastoma cell line CHP-134 and human myeloma cell line NCI-H929 at a fixed concentration of 10 micrograms per milliliter by flow cytometry as described in Moe et al, J of Exp & Clin Can Res 2021, 40:293. In brief, adherent cells were suspended in RPMI 1640 cell culture medium containing 10% (volume/volume) fetal bovine serum (FBS, ThermoFisher Scientific) by pipetting or treatment with ACCUTASE™ (Innovative Cell Technologies). The cells were centrifuged (200×g, 8 minutes) and the cell pellet suspended in medium to a concentration of 0.5-2×10\hat{ }6 cells per milliliter. The cells and antibodies were combined in 1.5 milliliter Eppendorf tubes and mixed by end over end rotation for 1 hour at ambient temperature. The tubes were centrifuged (200×g, 2 minutes), washed 1× in fresh medium and the cells suspended in medium containing AlexaFluor 488-conjugated goat anti-human F(ab′)2 secondary antibody. After end over end mixing for 30 minutes, the cells were washed 1× with fresh medium and the cells suspended in PBS buffer containing 0.5% (volume/volume) formaldehyde. Fluorescence of the cells was measured by flow cytometry (Acea NovoCyte). The results expressed as mean fluorescence intensity (MFI) over that of an irrelevant human IgG1 negative control antibody (BioXCell) are summarized in Table 1.

TABLE 1

| Antibody | CHP-134 MFI | NCI-H929 MFI |
|---|---|---|
| SAC-1 | 155,241 | 66,816 |
| SAC-2 | 59,426 | 67,142 |

### Example 2

The following example illustrates binding of an antibody provided herein to additional diverse cancer cell lines but not to CHP-134 neuroblastoma cells in which the polysialyltransferase genes ST8SIA2 and ST8SIA4 have been knocked out (CHP-134 KO). dPSA on the surface of CHP-134 cells depends on the expression of ST8SIA2 as described in Moe et al, J of Exp & Clin Can Res 2021, 40:293.

The antibody as provided in Example 1 was further tested for binding to multiple additional cell lines according to a similar procedure as that set forth in Example 1 with respect to CHP-134 and NCI-H929 cells. Table 2 sets for the EC₅₀ and maximum MFI values for each cell line, and shows that the antibody binds multiple diverse types of cancer cells. Furthermore, the antibody showed significantly enhanced binding as compared to a reference anti-dPSA antibody, SEAM 3 (Steirer and Moe et al., PLoS One 6(11): e27249 (2011)).

TABLE 2

| | SAC-1 | | SAC-2 | | SEAM 3 | |
|---|---|---|---|---|---|---|
| | EC₅₀ | | EC₅₀ | | EC₅₀ | |
| Cell Line | (nM) | MFIₘₐₓ | (nM) | MFIₘₐₓ | (nM) | MFIₘₐₓ |
| CHP-134 | 37.6 | 85,976 | 1.74 | 25,258 | 163 | 375,449 |
| CHP-134 KO | ND* | 1,150 | ND | 437 | ND | 10,262 |
| H929 | 32.1 | 10,609 | 7.8 | 9,940 | 115.7 | 371,201 |
| SK-MEL-28 | 23.2 | 12,296 | 2.2 | 17,934 | 2885 | 80,152 |
| AsPC-1 | Negative | — | 0.76 | 71,109 | Negative | — |
| BxPC-3 | 2.7 | 3,617 | 2.24 | 122,253 | Negative | — |
| NCI-N87 | 6.3 | 14,346 | 1.3 | 27,126 | ND | ND |
| SNU-16 | 2.4 | 3,988 | 6.4 | 5,088 | 199 | 45,646 |
| SK-OV-3 | 1 | 3,995 | 0.62 | 231,541 | Negative | — |

*ND, not determined.

### Example 3

The following example illustrates that the antibodies provided herein bind a different epitope than a reference antibody.

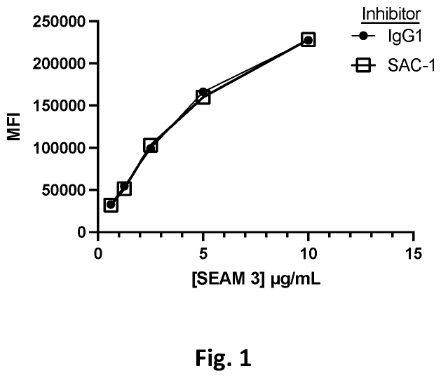

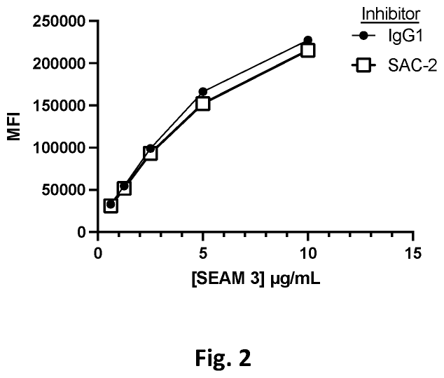

To determine whether the epitope recognized by the antibodies of Example 1 are distinct from that recognized by reference anti-dPSA antibody, SEAM 3 (Steirer and Moe et al., PLoS One 6(11): e27249 (2011)), a fixed concentration of antibody 10× greater than the EC50 for binding to CHP-134 cells was combined with serial 2-fold dilutions of SEAM 3. As shown in FIG. 1 and FIG. 2, the antibodies of Example 1 did not inhibit SEAM 3 binding, which demonstrates that the antibodies recognize different epitopes.

### Example 4

This Example demonstrates that the antibodies provided herein bind to multiple human cancer cell lines.

Cell lines were obtained from American Type Culture Collection (ATCC, Manassas, VA) and were routinely tested for mycoplasma contamination (MycoStrip™, InvivoGen, San Diego, CA). Cell lines were grown in medium recommended by ATCC in a humidified chamber in an atmosphere of 5% CO2. Adherent cell lines were suspended by treatment with StemPro™ Accutase™ cell dissociation reagent (Thermo Fisher Scientific, Carlsbad, CA). The suspended cells were diluted 1:5 with cell culture medium, centrifuged (200×g, 10 minutes), and suspended in fresh medium to a density of 1-10 million viable cells/mL. Viability was determined by Trypan Blue staining (Thermo Fisher Scientific, Carlsbad, CA) and the cell counted using SKC, Inc. C-Chip™ Disposable Hemacytometers (Fisher Scientific, Pittsburgh, PA). The cells and antibodies (SAC-1, SAC-1 humanized (SAC-1.1), SAC-2, SAC-2 mouse (SEQ ID NO: 54 and SEQ ID NO: 55), SAC-2 humanized C (SAC-2.2), and SAC-2 humanized D (SAC-2.1) were combined in tubes incubated at ambient temperature for 1 hour with end over end continuous mixing. The cells were centrifuged (200×g, 2 minutes), the supernatant removed by aspiration and cells suspended in medium containing the appropriate (i.e. anti-mouse or anti-human IgG H+L) secondary antibody labeled with Alexa Fluor 488™ (AffiniPure F(ab′)2 Fragment Goat Anti-Mouse IgG (H+L), Jackson ImmunoResearch, West Grove, PA). The cells and secondary antibody were incubated at ambient temperature with end over end mixing for 30 minutes then centrifuged (200×g, 2 minutes), supernatant removed by aspiration, and the cells suspended in phosphate buffered saline containing 0.5% formaldehyde.

Finally, the cells were analyzed by flow cytometry (Acea NovoCyte, Agilent, Santa Clara, CA or similar). Binding curves for the dependence of mean fluorescence intensity (MFI) on antibody concentration were analyzed curve fitting software (GraphPad Prism, San Diego, CA) to determine the binding constant (K_{D}) and maximum MFI (MFImax). The results are presented in Table 3 (SAC-1 antibodies) and Table 4 (SAC-2 antibodies), wherein Neg. means negative and NA means not applicable. As shown in Tables 3 and 4, the tested antibodies bind to cell lines from multiple cancers with K_{D} in the nanomolar range and have variable MFImax values depending on the epitope density of each cell line. This data shows the antigen recognized by the antibodies is present on a wide range of human cancers and, since knocking out the gene coding for the polysialyl transferase, STSIA2, in CHP-134 cells eliminates binding, is dependent on expression of that enzyme.

TABLE 3

| | | SAC-1 Chimera | | SAC-1 Humanized | |
|---|---|---|---|---|---|
| | | K_{D} | | K_{D} | |
| Cancer | Cell line | (nM) | MFIₘₐₓ | (nM) | MFIₘₐₓ |
| Neuroblastoma | CHP-134 | 3.5 | 24,063 | — | — |
| | CHP-134 (dPSA KO) | NA | 939 | — | — |
| Melanoma | SK-N-MC | — | — | — | — |
| | SK-MEL-28 | 37.3 | 10,162 | — | — |
| | A375 | 3.3 | 8,378 | 14.5 | 55,394 |
| Pancreatic | AsPC-1 | NA | Neg. | — | — |
| | BxPC-3 | 2.7 | 3,617 | 1.7 | 6,609 |
| | Panc1 | 0.3 | 17,740 | — | — |
| Gastric | NCI-N87 | 6.3 | 14,346 | 3.2 | 13,655 |
| | SNU-16 | 2.4 | 3,988 | | |
| Ovarian | SK-OV-3 | 1 | 3,995 | 0.9 | 3,914 |
| Breast | MDA-MB-231 | 2 | 35,007 | — | — |
| Cervical | ME-180 | NA | Neg. | — | — |
| Plasmacytoma | NCI-H929 | 32.1 | 10,609 | — | — |
| Lymphoma | Jeko-1 | NA | Neg. | — | — |
| Lung | NCI-H526, SCLC (E) | — | — | 29.1 | 12,128 |
| | NCI-H524, SCLC (L) | — | — | 114.5 | 360,150 |
| | NCI-H69, carcinoma | — | — | 64.7 | 134,103 |
| | NCI-H549, NSCLC | — | — | 79.6 | 1,863 |
| | NCI-H1395, | — | — | 161.7 | 214,210 |
| | adenocarcinoma | | | | |

TABLE 4

| | | | | | | SAC-2 | | SAC-2 | |
|---|---|---|---|---|---|---|---|---|---|
| | | SAC-2 Chimera | | SAC-2 Mouse | | Humanized C | | Humanized D | |
| | | K_{D} | | K_{D} | | K_{D} | | K_{D} | |
| Cancer | Cell line | (nM) | MFIₘₐₓ | (nM) | MFIₘₐₓ | (nM) | MFIₘₐₓ | (nM) | MFIₘₐₓ |
| Neuroblastoma | CHP-134 | 1.3 | 64,334 | — | — | — | — | — | — |
| | CHP-134 | NA | 640 | — | — | — | — | — | — |
| | (dPSA KO) | | | | | | | | |
| Melanoma | SK-N-MC | — | — | — | — | — | — | — | — |
| | SK-MEL-28 | 3.8 | 252,551 | — | — | — | — | — | — |
| | A375 | 0.7 | 595,028 | 1.3 | 405.309 | 1.52 | 614,998 | 1.40 | 713,551 |
| Pancreatic | AsPC-1 | 0.76 | 71,109 | — | — | — | — | — | — |
| | BxPC-3 | 2.2 | 122,253 | 0.9 | 87.351 | — | — | — | — |
| | Panc1 | 1.2 | 1,217,377 | — | — | — | — | — | — |
| Gastric | NCI-N87 | 1.3 | 27,126 | 3.2 | 99,214 | — | — | — | — |
| | SNU-16 | 6.4 | 5,088 | — | — | — | — | — | — |
| Ovarian | SK-OV-3 | 0.62 | 231,541 | 0.9 | 202,657 | — | — | — | — |
| Breast | MDA-MB-231 | 6 | 945,970 | — | — | 1.90 | 658,525 | 1.78 | 737,731 |
| Cervical | ME-180 | 0.2 | 23,010 | — | — | — | — | — | — |
| Plasmacytoma | NCI-H929 | 0.35 | 36,419 | — | — | 0.72 | 38,901 | 0.77 | 43,514 |
| Lymphoma | Jeko-1 | 1.7 | 63,451 | — | — | — | — | — | — |
| Lung | NCI-H526, | — | — | 13 | 1,287 | — | — | — | — |
| | SCLC (E) | | | | | | | | |
| | NCI-H524, | — | — | NA | Neg. | — | — | — | — |
| | SCLC (L) | | | | | | | | |
| | NCI-H69, | — | — | 0.58 | 114,556 | — | — | — | — |
| | carcinoma | | | | | | | | |
| | NCI-H549, | — | — | 4.1 | 746,498 | — | — | — | — |
| | NSCLC | | | | | | | | |
| | NCI-H1395, | — | — | 4.2 | 12,628 | — | — | — | — |
| | adenocarcinoma | | | | | | | | |

### Example 5

This Example demonstrates that the antibodies provided herein recognize a deriviatives of nucleolin comprising dPSA.

Preparation of subcellular fractions: Human melanoma A375 cells (80% confluent in a T-175 flask) were extracted using the ProteoExtract® Subcellular Proteome Extraction Kit (MilliporeSigma). In brief, the differential detergent extraction procedure used four extraction buffers sequentially, along with a protease inhibitor cocktail to prevent protein degradation during the extraction and Benzonase® nuclease (Sigma-Aldrich, St. Louis, MO) to degrade contaminating nucleic acids. The manufacturer's instructions for extraction were followed, and the cell extracts were separated into four fractions: F1 (cytosolic fraction), F2 (cell membrane fraction), F3 (nucleic protein fraction), and F4 (cytoskeletal fraction).

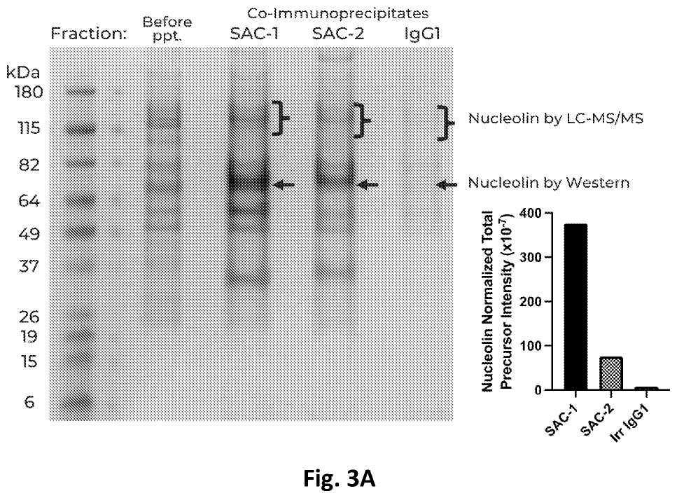

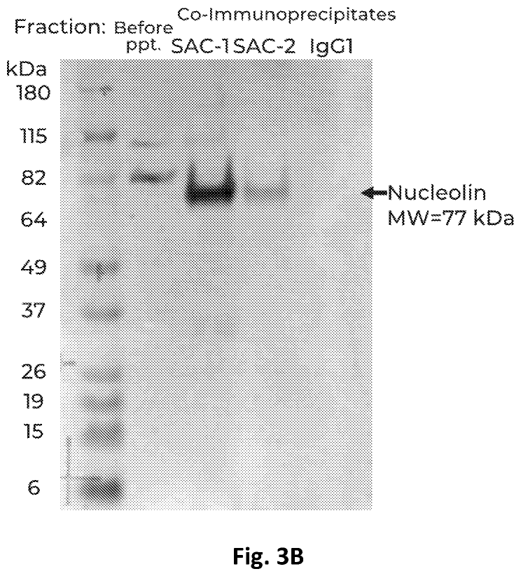

Co-immunoprecipitation: Dynabeads M-270 epoxy magnetic beads (Thermo Fisher Scientific, Carlsbad, CA) covalently linked to SAC-1, SAC-2, or an irrelevant human IgG1 antibody (BioXCell, Lebanon, NH) were prepared following the manufacturer's protocol. Antigens reactive with the antibodies were purified through co-immunoprecipitation as follows. The F2 membrane fraction was incubated separately with SAC-1, SAC-2 or irrelevant IgG1-linked magnetic beads. The beads were separated using a magnet, washed with the respective extraction buffer alone, and then with buffer containing polysialic acid (polySia) (50 pg/mL; colominic acid from Sigma-Aldrich, St. Louis, MO) to remove nonspecific binding antigens. Finally, SDS-PAGE sample buffer (NuPAGE, Thermo Fisher Scientific) without reducing agent was used to elute the antigens from the beads by heating to 80° C. for 10 minutes. Proteins eluted from the beads were resolved on 4%-12% SDS-PAGE (NuPAGE, Thermo Fisher Scientific) and either stained with SimplyBlue® Coomasie stain (Thermo Fisher Scientific) or transferred to a PVDF membrane (Immobilon®-FL, Millipore, Waltham, MA) using a NuPAGE transfer cell (Thermo Fisher Scientific) for Western blotting. The PVDF membrane was blocked overnight with 5% (weight/volume) dry whole milk in phosphate buffered saline (PBS) buffer then the immunoprecipitates were stained with anti-nucleolin antibody MS-3 (Santa Cruz Biotechnology, Santa Cruz, CA) in blocking buffer for 2 hours at ambient temperature. After washing three times with PBS buffer the bound antibody was detected with IRDye® 800CW-conjugated donkey anti-mouse IgG (H+L) secondary antibody (LI-COR, Lincoln, NE). Images of gels and blots were recorded on an Odyssey® Fc Imaging System (LI-COR). The results are presented in FIGS. 3A and 3B.

FIG. 3A shows proteins co-immunoprecipitated by each antibody resolved on an SDS-PAGE. There are multiple, strongly staining bands that have the same distribution of apparent mass co-immunoprecipitated by SAC-1 and SAC-2 but not by the irrelevant IgG1 antibody. Previously, we showed that antibodies that bind to de-N-acetyl polysialic acid (dPSA) co-immunoprecipitate nucleolin modified with dPSA from the F2 fraction of cancer cells (J Exp Clin Cancer Res. 2021 Sep. 20; 40(1):293. Cell surface nucleolin is unique to cancer cells and has a range of apparent mass when resolved on SDS-PAGE gels because of multiple post-translational modifications and length and charge heterogeneity of dPSA.

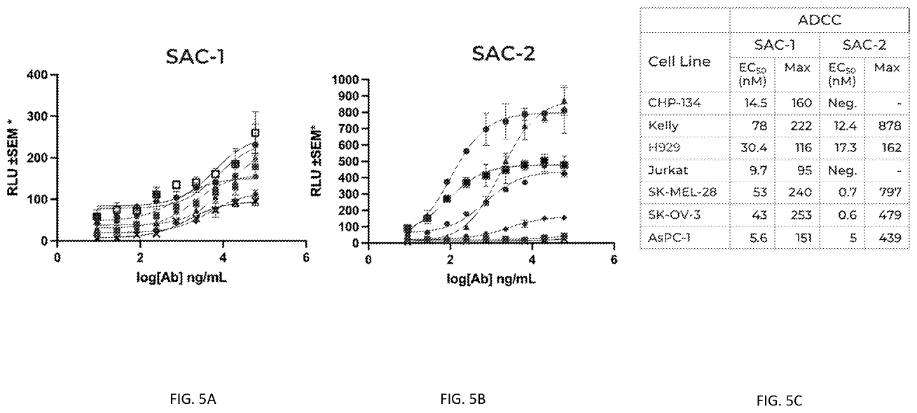

FIG. 3B shows staining of the co-immunoprecipitated proteins with anti-nucleolin antibody MS-3 on a Western blot. The major band for SAC-1 and SAC-2 samples (i.e. band with apparent mass of 77 kDa) is nucleolin based on reactivity with MS-3. The post-translational modifications of nucleolin that affect migration in SDS-PAGE gels may not be detected by MS-3, which was made to unmodified, recombinant nucleolin. To confirm that other bands having apparent masses different from the major band detected with MS-3 were also derivatives of nucleolin, sections of the gel indicated by brackets in FIG. 5A were cut out for each of the co-immunoprecipitated samples and processed as described previously (J Exp Clin Cancer Res. 2021 Sep. 20; 40(1):293) for analysis by LC-MS/MS mass spectroscopy for protein identification. LC-MS/MS mass spectroscopy confirmed that the section of the gel analyzed also contained nucleolin.

The quantification of nucleolin for each sample also is shown in FIG. 5A and corresponds to the relative amount of Coomassie staining in that section of the SDS-PAGE gel for each sample. In summary, the data show that SAC-1 and SAC-2 bind to the same modified form of nucleolin located in the membrane fraction of A375 human melanoma cells and that derivative of nucleolin has characteristics (i.e., a range of apparent mass on SDS-PAGE) of being modified with dPSA as described above.

### Example 6

This Example demonstrates that the antibodies provided herein exhibit reactivity with human tumors and normal human tissues by immunohistochemistry.

To determine the specificity of the SAC antibodies for binding to cancer cells but not normal post-development human tissues, SAC-2 was used to stain tissue micro arrays containing tissue specimens from normal human tissues and tumors by immunohistochemistry.

SAC-2 mouse monoclonal antibody (SEQ ID NO: 54 and SEQ ID NO: 55) was used at 2.5 micrograms/milliliter with Tris-based pH 9.5 Heat-Induced Epitope Retrieval; an isotype control (mouse IgG2a) was used under the same conditions. Formalin fixed paraffin embedded (FFPE) sections were stained on the Biocare intelliPATH automated staining platform (Biocare Medical, Pacheco, CA) using the manufacturer's recommended settings. The sections were incubated with Biocare Peroxidase Blocker (Biocare, Cat. #PX968) and Background Punisher (Biocare, Cat. #BP974M) to block non-specific background. For the detection of mouse primary antibodies, MACH4 HRP-polymer Detection System (Biocare, Cat. #MRH534) was used. The chromogenic detection and counterstaining kits IntelliPATH FLX DAB chromogen (Biocare, Cat. #IPK5010) and IntelliPATH Hematoxylin (Biocare Medical, Cat. #XMF963) were used. Tissue micro arrays (TMAs) were from Pantomics (Fairfield, CA). The TMAs included the FDA recommended panel of normal human tissues from three different donors (MNO961). The TMAs for human tumors included a multi-tumor array (MTU481) and arrays for breast (BRC1022), colorectal (COC1021), lung (primary and metastatic, LUM961), metastatic (MET961), ovarian (OVC1021), and pancreatic (PAN1021) cancer, and lymphoma (LYM1021). The TMA slides were stained with the mouse version of the SAC-2 antibody to eliminate background resulting from secondary antibody binding to human IgG in the samples. Stained slides were digitized with a TissueScope LE whole slide scanner (Huron Digital Pathology, St. Jacobs, Ontario, Canada) at 20× magnification. For the purpose of confirmation, glass slides were examined on an upright bright-field microscope.

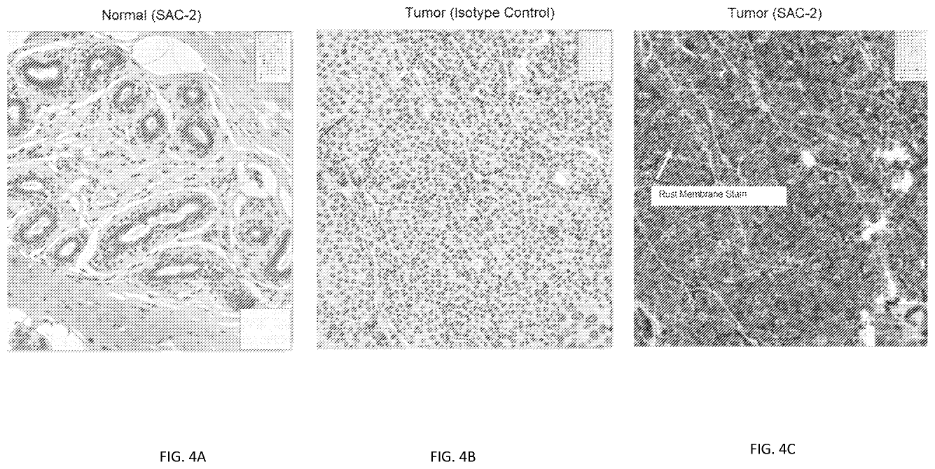

FIG. 4 shows an example of SAC-2 staining of normal human breast tissue (FIG. 4A) and a breast tumor (FIG. 4C) compared with staining of the tumor by an irrelevant mouse IgG2a antibody (FIG. 4B). Staining of the tumor is shown by the rust-brown membrane staining of tumor cells while the normal breast tissue is not stained by SAC-2 and the tumor is not stained by the irrelevant IgG2a antibody. SAC-2 staining of the breast tumor cells was homogenous throughout the specimen. Although the intensity of SAC-2 staining of tumor cells was variable, for different specimens, the homogeneous staining characteristic was consist in all specimens that were positive for SAC-2 binding.

Table 5 summarizes the staining results for normal human tissues. The only positive staining of normal human tissues observed was one of three prostate specimens. The stained prostate sample was described as benign prostate hyperplasia, which may have also contained early-stage prostate cancer cells. Many of the tumor specimens, in particular, metastatic tumors were stained. Table 6, summarizes the results for staining the tumor tissue microarrays with SAC-2. In summary, the results show that SAC-2 does not bind to normal human post-development tissues but does recognize antigens expressed on cells of several different human primary and metastatic tumors.

TABLE 5
Summary of SAC-2 staining on normal human tissues
(Pantomics MNO96) by immunohistochemistry.

| Tissue | Staining positive |
|---|---|
| Adrenal gland | 0 of 3 |
| Bladder | 0 of 3 |
| Bone marrow | 0 of 1 |
| Eye | 0 of 2 |
| Breast | 0 of 3 |
| Brain, cerebellum | 0 of 3 |
| Brain, cerebral cortex | 0 of 3 |
| Fallopian tube | 0 of 3 |
| Esophagus | 0 of 3 |
| Stomach | 0 of 3 |
| Intestine, sm | 0 of 3 |
| Intestine, colon | 0 of 3 |
| Intestine, rectum | 0 of 3 |
| Heart | 0 of 3 |
| Kidney | 0 of 5 |
| Kidney, cortex | 0 of 1 |
| Liver | 0 of 3 |
| Lung | 0 of 3 |
| Ovary | 0 of 3 |
| Pancreas | 0 of 3 |
| Parathyroid | 0 of 1 |
| Pituitary | 0 of 2 |
| Placenta | 0 of 3 |
| Prostate | 1 of 3* |
| Skin | 0 of 2 |
| Spinal cord | 0 of 2 |
| Spleen | 0 of 2 |
| Skeletal muscle | 0 of 3 |
| Testis | 0 of 3 |
| Thymus | 0 of 3 |
| Thyroid | 0 of 3 |
| Tonsil | 0 of 3 |
| Ureter | 0 of 3 |
| Uterus, cervix | 0 of 3 |
| Uterus, endometrium | 0 of 3 |

*Specimen stained was one of three and was described as benign prostate hyperplasia.

TABLE 6
Summary of SAC-2 immunohistochemical staining
of tumors specimens on tissue micro arrays.

| | Percent positive | |
|---|---|---|
| Patient tumor panels | (number positive/total | |
| (Pantomics Cat. No.) | number of samples | |
| 15 different cancer types (MTU481) | 46% | (22/48) |
| Breast (BRC1022) | 78% | (62/80) |
| Lung (LUM961) | 73% | (73/100) |
| Colorectal (COCR1021) | 20% | (20/100) |
| Lymphoma (LYM1021) | 17% | (15/88) |
| Pancreatic (PAN1021) | 21% | (19/88) |
| Ovarian (OVC1021) | 0% | (0/96) |
| Metastatic (MET961) | 81% | (73/90) |

### Example 7

This Example demonstrates that the antibodies provided herein have antibody-dependent cellular cytotoxicity activity.

Five adherent cell lines (CHP-134 and Kelly neuroblastoma, SK-MEL-28 melanoma, SK-OV-3 ovarian, and AsPC-1 pancreatic) and two non-adherent (NCI-H020 myeloma, Jurkat leukemia) cancer cell lines were tested with SAC-1 and SAC-2 for the ability to mediate ADCC activity in vitro. Cell lines were obtained and prepared for use in the assay as described above in Example 4. ADCC activity was measured using the InvivoGen Jurkat-Lucia™ NFAT-CD16 reporter assay kit following the manufacturer's instructions. The assay measures the cell killing CD-16Fcgamma receptor-mediated signaling pathway activated by antibody binding to antigens on the surface of the cancers and binding of the Fc portion of the antibody to receptors on the reporter cells resulting in expression of luciferase and the production of luminescence in the presence of luciferin. The output signal as relative luminescence units (RLU), was measured using a luminescence plate reader (Synergy HTX Multimode Reader, Agilent, Santa Clara, CA or similar).

The results for mouse-human Fc IgG1 chimera versions of SAC-1 and SAC-2 are shown in the graphs of FIGS. 5A-5C. In general, SAC-1 had less signal than SAC-2, reflecting the lower epitope density reflected in the relative MFI determined in binding studies for the antibody (Table 3), but was able to activate ADCC activity against all cell lines tested. SAC-2 had higher ADCC activity against most cell lines tested, which also is consistent with the higher density of epitopes observed in binding studies (Table 4), but SAC-2 lacked activity against some cell lines. In summary, the data show that both antibodies can mediate ADCC activity against multiple different human cancer cell lines.

### Example 8

This Example demonstrates that the antibody-dependent cellular cytotoxicity (ADCC) activity of the humanized SAC-2.1C and D antibodies is enhanced by reducing or eliminating fucosylation (e.g., afucosylated antibodies).

The ADCC activity of the antibodies with reduced fucosylation (aFUC) was compared using the DELFIA® cell cytotoxicity assay kit (PerkinElmer, Billerica, MA). The target cells used were human A375 melanoma and MDA-MB-231 breast cancer cells. The assay was performed using the manufacturers instructions. In brief, the target cells were harvested and suspended in complete medium. The target cells (1×10\hat{ }6) were labeled with 2 uL fluorescence enhancing ligand (DELFIA@BATDA Reagent) for 20 min at 37° C. The cells were washed 4 times with phosphate buffered saline (PBS) then the cell density was adjusted to 1×10\hat{ }5/mL and seeded 100 mL/well to 96-well assay plates. In the ADCC assay, 50 sL of serially diluted antibody solutions were added into wells, and incubated at 37° C., 5% CO2 for 15 min. Natural killer effector cells (NK92/CD16a) were added at a ratio of 8 effector cells to 1 target cells in 50 sL/well of the 96-well plate. The assay plates with effector cells, antibodies and target cells were incubated at 37° C., 5% CO2 for 3 hours.

While cells are intact, BATDA ligand remains in the cell. When Europium solution is added to supernatant from a sample of intact cells, the Europium is unable to form a fluorescent chelate with BATDA, as no BATDA is released into the supernatant. Europium solution is not fluorescent in its unaltered state. If cells are lysed by an effector cell, BATDA is released outside the cell into the supernatant. Upon addition of Europium solution to the supernatant, Europium can form a highly fluorescent and stable chelate with the released BATDA (EuTDA). The measured fluorescence signal correlates directly with the amount of lysed cells in the cytotoxicity assay.

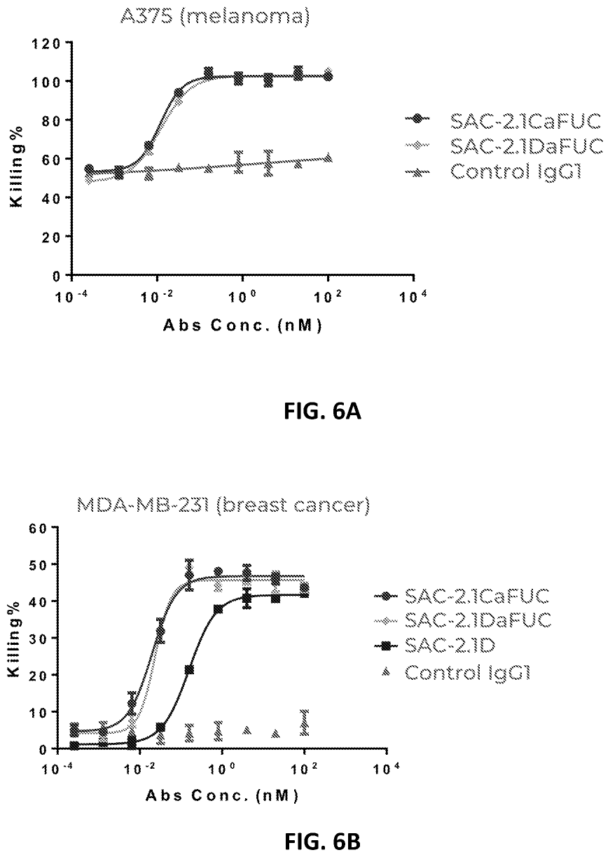

To determine the maximum release of BATDA, 10 μL of Lysis Buffer was added to control wells and 20 μL of the supernatant from all wells was transferred to a flat-bottom detection plate. Europium Solution (200 μL) was added to the supernatant in the flat well plate, the plate was shaken for 15 min at room temperature and, finally, the fluorescence was measured in a time-resolved fluorometer within 5 hours. The percent killing was calculated as: ADCC=(Fluorescence of sample−Spontaneous fluorescence of target cell and effect cell mixture/(Fluorescence of target cell Maximum−Spontaneous fluorescence of target cell and effect cell mixture)×100%. The percent killing curves were analyzed to determine the EC50 using GraphPad Prism 6 (GraphPad, San Diego, CA). The results are shown in FIGS. 6A and 6B and summarized in Table 7. Depending on the antibody tested and the target cells, the ADCC activity increased by 4- to 10-fold.

TABLE 7

| | A375 cells | | Ratio | MDA-MB-231 cells | | Ratio |
|---|---|---|---|---|---|---|
| Antibodies | EC50 (nM) | Span | aFUC/FUC | EC50 (nM) | Span | aFUC/FUC |
| SAC-2.1C | 0.1 | 56 | 10 | 0.16 | 38 | 8 |
| SAC-2.1C/aFUC | 0.01 | 49 | | 0.02 | 42 | |
| SAC-2.1D | 0.04 | 56 | 4 | 0.16 | 40 | 8 |
| SAC-2.1D/aFUC | 0.01 | 54 | | 0.02 | 41 | |

### Example 9

This Example demonstrates that the antibodies provided herein have in vivo tumor inhibition activity in xenograft mouse models of human cancer.

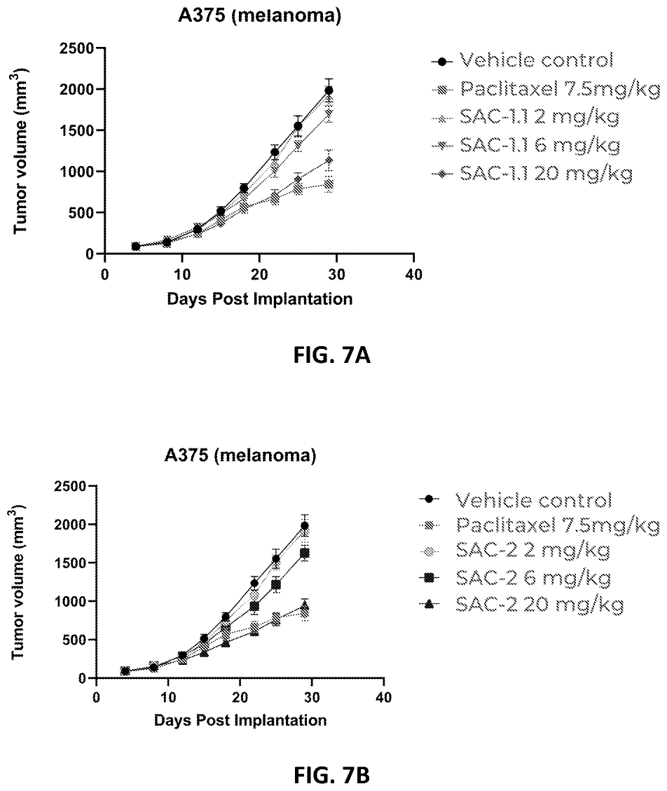

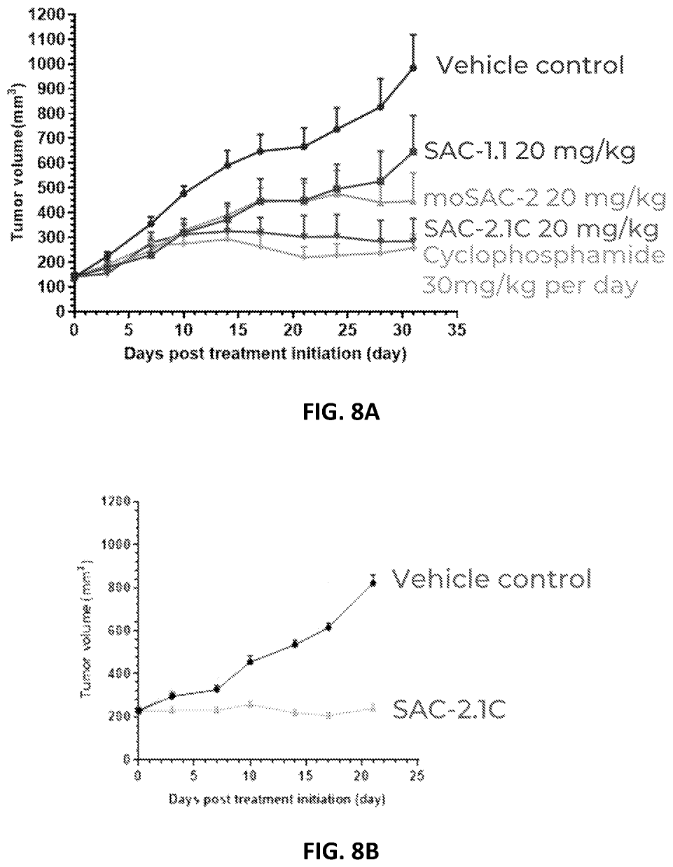

The ability of the SAC antibodies to inhibit tumor growth in xenograft mouse models of human cancer were tested using two models, A375 human melanoma and MDA-MB-231 human breast cancer, in athymic BALB/c nu/nu mice and in NSG mice supplemented with human peripheral blood mononuclear cells (PBMCs). The mice were inoculated with a subcutaneous injection of 1×10\hat{ }6 cells plus Matrigel in the hind leg. The tumors were calipered (digital calipers) and grouped in treatment cohorts (10 mice per group) with an average size of 100-200 mm³. The mice were treated with SAC antibodies at 2 mg/kg, 6 mg/kg and 20 mg/kg two times per week or standard of care drugs (paclitaxel 7.5 mg/kg for the A375 model; cyclophosphamide 30 mg/kg, i.p. daily for the MDA-MB-231 model) or vehicle alone for the control group. Mice given human PBMCs were treated one time with the PBMCs at the start of the treatment period. Comparisons of significance were made by repeated measures ANOVA with Dunnett's Multiple Comparison Test using GraphPad Prism (San Diego, CA) software. The dose-dependence of SAC antibody treatment compared to standard of care paclitaxel is shown in FIGS. 7A and 7B. The data for SAC-1.1, mouse SAC-2, and SAC-2.1C in the MDA-MB-231 human breast cancer xenograft mouse model is shown in FIG. 8A and the effect of adding human PBMCs with SAC-2.1C is shown in FIG. 8B. The data show that SAC-1 and SAC-2 can inhibit tumor growth in xenograft mouse models of human melanoma and breast cancer that depends on concentration of the antibodies.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A dPSA-binding agent comprising
(a) an immunoglobulin heavy chain variable region comprising SEQ ID NO: 51 or at least the complementarity determining regions (CDRs) thereof; and an immunoglobulin light chain variable region comprising SEQ ID NO: 52 or 53 or at least the CDRs thereof;
(b) an immunoglobulin heavy chain variable region comprising any of SEQ ID NOs: 17-20 or at least the complementarity determining regions (CDRs) thereof; and an immunoglobulin light chain variable region comprising SEQ ID NO: 21 or at least the CDRs thereof;
(c) an immunoglobulin heavy chain variable region comprising any of SEQ ID NOs: 1-4 or at least the complementarity determining regions (CDRs) thereof; and an immunoglobulin light chain variable region comprising SEQ ID NO: 5 or at least the CDRs thereof;
or (d) an immunoglobulin heavy chain variable region comprising SEQ ID NO: 35 or at least the complementarity determining regions (CDRs) thereof; and an immunoglobulin light chain variable region comprising SEQ ID NO: 36 or at least the CDRs thereof.

2. A dPSA binding agent comprising
(a) an immunoglobulin heavy chain variable region comprising:
CDRH1 comprising any one of SEQ ID NOs: 6-9;
CDRH2 comprising SEQ ID NO: 10;
CDRH3 comprising SEQ ID NO: 11;
and an immunoglobulin light chain variable region comprising
CDRL1 comprising SEQ ID NO: 12;
CDRL2 comprising SEQ ID NO: 13; and
CDRL3 comprising SEQ ID NO: 14;
(b) an immunoglobulin heavy chain variable region comprising:
CDRH1 comprising any one of SEQ ID NOs: 24-27;
CDRH2 comprising SEQ ID NO: 28;
CDRH3 comprising SEQ ID NO: 29;
and an immunoglobulin light chain variable region comprising
CDRL1 comprising SEQ ID NO: 30;
CDRL2 comprising SEQ ID NO: 31; and
CDRL3 comprising SEQ ID NO: 32;
(c) an immunoglobulin heavy chain variable region comprising:
CDRH1 comprising SEQ ID NO: 39;
CDRH2 comprising SEQ ID NO: 40; and
CDRH3 comprising SEQ ID NO: 41 or 47;
and an immunoglobulin light chain variable region comprising
CDRL1 comprising SEQ ID NO: 42;
CDRL2 comprising SEQ ID NO: 43 or 56; and
CDRL3 comprising SEQ ID NO: 44;
or (d) an immunoglobulin heavy chain variable region comprising:
CDRH1 comprising SEQ ID NO: 45;
CDRH2 comprising SEQ ID NO: 46; and
CDRH3 comprising SEQ ID NO: 41 or 47;
and an immunoglobulin light chain variable region comprising
CDRL1 comprising SEQ ID NO: 48;
CDRL2 comprising SEQ ID NO: 49 or 56; and
CDRL3 comprising SEQ ID NO: 50.

3. The dPSA-binding agent of claim 1, wherein the dPSA-binding agent is an antibody, an antigen-binding antibody fragment, a conjugate thereof, or a chimeric antigen receptor.

4. The dPSA-binding agent of claim 1, wherein the dPSA-binding agent is a F(ab′)₂ fragment, a Fab′ fragment, a Fab fragment, a Fv fragment, a scFv fragment, a dsFv fragment, or a dAb fragment.

5. The dPSA binding agent of claim 1, wherein the dPSA-binding agent comprises an Fc region that can mediate complement dependent cytotoxicity (CDC) or antibody-dependent cellular cytotoxicty (ADCC).

6. The dPSA binding agent of claim 1, wherein the dPSA-binding agent is an IgG1 or IgG4 antibody.

7. The dPSA binding agent of claim 1, wherein the dPSA binding agent is afucosylated.

8. A composition comprising (a) the dPSA-binding agent of claim 1 or nucleic acid encoding same and (b) a pharmaceutically acceptable carrier.

9. A nucleic acid encoding the immunoglobulin heavy chain and/or light chain variable region of the dPSA-binding agent of claim 1.

10. The nucleic acid of claim 9, further encoding a leader sequence for the immunoglobulin heavy chain and/or light chain variable region.

11. A cell comprising the nucleic acid of claim 9.

12. A cell line that expresses the dPSA binding agent of claim 1.

13. A method of preparing a dPSA-binding agent according to claim 1, the method comprising expressing a nucleotide sequence encoding the immunoglobulin heavy chain polypeptide and a nucleic acid sequence encoding the immunoglobulin light chain polypeptide in a cell.

14. A method of killing a cancer cell that expresses dPSA, the method comprising contacting the cancer cell with a dPSA binding agent of claim 1.

15. A method of treating cancer characterized by dPSA expression in a subject, the method comprising administering to the subject a dPSA-binding agent of claim 1.

16. A method of delivering a payload to a cell that expresses dPSA, the method comprising contacting the cell with a dPSA binding agent of claim 1 conjugated to the payload.

17. The method of claim 16, wherein the payload is a cytotoxic molecule or a detectable label.

18. The method of claim 16, wherein the cell is a cancer cell.

19. A method of detecting soluble dPSA in a biological fluid or tissue sample comprising contacting the biological fluid or tissue sample with dPSA binding agent of claim 1, optionally conjugated to a detectable label.

20. The method of claim 19, wherein the method is used to detect cancer or to select a patient for treatment with a dPSA binding agent.

21.-22. (canceled)


SEQUENCE LISTING (59 sequences)

SEQ ID NO: 1 - protein, 120 aa, synthetic construct
EVQLVESGGGLVQPKGSLKLSCAASGFSFNTYAMNWVRQAPGKGLEWVARIRSKSNNYATYYADSVKDRFTISRDDSESMLYLQMNNLKTEDTAIYYCVRQVTGLAMDYWGQGTSVTVSS

SEQ ID NO: 2 - protein, 120 aa, synthetic construct
EVQLVESGGGLVQPKGSLKLSCAASGFTFNTYYMNWVRQAPGKGLEWVARIRSKSNNYATYYADSVKDRFTISRDDSESMLYLQMNNLKTEDTAIYYCVRQVTGLAMDYWGQGTSVTVSS

SEQ ID NO: 3 - protein, 120 aa, synthetic construct
EVQLVESGGGLVQPKGSLKLSCAASGFEFNTYAMNWVRQAPGKGLEWVARIRSKSNNYATYYADSVKDRFTISRDDSESMLYLQMNNLKTEDTAIYYCVRQVTGLAMDYWGQGTSVTVSS

SEQ ID NO: 4 - protein, 120 aa, synthetic construct
EVQLVESGGGLVQPKGSLKLSCAASGFEFNTYYMNWVRQAPGKGLEWVARIRSKSNNYATYYADSVKDRFTISRDDSESMLYLQMNNLKTEDTAIYYCVRQVTGLAMDYWGQGTSVTVSS

SEQ ID NO: 5 - protein, 112 aa, synthetic construct
DIVMTQAAPSVPVTPGESVSISCRSSKSLLYSNGNTYLYWFLQRPGQSPQLLIYRMSNLASGVPDRFSGSGSGTAFTLRISRVEAEDVGVYYCMQHLEYPLTFGAGTKLELK

SEQ ID NO: 6 - protein, 9 aa, synthetic construct
GFSFNTYAM

SEQ ID NO: 7 - protein, 9 aa, synthetic construct
GFTFNTYYM

SEQ ID NO: 8 - protein, 9 aa, synthetic construct
GFEFNTYAM

SEQ ID NO: 9 - protein, 9 aa, synthetic construct
GFEFNTYYM

SEQ ID NO: 10 - protein, 10 aa, synthetic construct
IRSKSNNYAT

SEQ ID NO: 11 - protein, 6 aa, synthetic construct
QVTGLA

SEQ ID NO: 12 - protein, 11 aa, synthetic construct
KSLLYSNGNTY

SEQ ID NO: 13 - skipped

SEQ ID NO: 14 - protein, 9 aa, synthetic construct
MQHLEYPLT

SEQ ID NO: 15 - protein, 450 aa, synthetic construct
EVQLVESGGGLVQPKGSLKLSCAASGFSFNTYAMNWVRQAPGKGLEWVARIRSKSNNYATYYADSVKDRFTISRDDSESMLYLQMNNLKTEDTAIYYCVRQVTGLAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 219 aa, synthetic construct
DIVMTQAAPSVPVTPGESVSISCRSSKSLLYSNGNTYLYWFLQRPGQSPQLLIYRMSNLASGVPDRFSGSGSGTAFTLRISRVEAEDVGVYYCMQHLEYPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 17 - protein, 120 aa, synthetic construct
EVQLVESGGGLVQPGGSLRLSCAASGFSFNTYAMNWVRQAPGKGLEWVARIRSKSNNYATYYADSVKDRFTISRDDSKNSLYLQMNSLKTEDTAVYYCVRQVTGLAMDYWGQGTTVTVSS

SEQ ID NO: 18 - protein, 120 aa, synthetic construct
EVQLVESGGGLVQPGGSLRLSCAASGFTFNTYYMNWVRQAPGKGLEWVARIRSKSNNYATYYADSVKDRFTISRDDSKNSLYLQMNSLKTEDTAVYYCVRQVTGLAMDYWGQGTTVTVSS

SEQ ID NO: 19 - protein, 120 aa, synthetic construct
EVQLVESGGGLVQPGGSLRLSCAASGFEFNTYAMNWVRQAPGKGLEWVARIRSKSNNYATYYADSVKDRFTISRDDSKNSLYLQMNSLKTEDTAVYYCVRQVTGLAMDYWGQGTTVTVSS

SEQ ID NO: 20 - protein, 120 aa, synthetic construct
EVQLVESGGGLVQPGGSLRLSCAASGFEFNTYYMNWVRQAPGKGLEWVARIRSKSNNYATYYADSVKDRFTISRDDSKNSLYLQMNSLKTEDTAVYYCVRQVTGLAMDYWGQGTTVTVSS

SEQ ID NO: 21 - protein, 112 aa, synthetic construct
DIVMTQSPLSLPVTPGEPVSISCRSSKSLLYSNGNTYLYWFLQKPGQSPQLLIYRMSNLASGVPDRFSGSGSGTAFTLKISRVEAEDVGVYYCMQHLEYPLTFGQGTKLEIK

SEQ ID NO: 22 - protein, 450 aa, synthetic construct
EVQLVESGGGLVQPGGSLRLSCAASGFSFNTYAMNWVRQAPGKGLEWVARIRSKSNNYATYYADSVKDRFTISRDDSKNSLYLQMNSLKTEDTAVYYCVRQVTGLAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 23 - protein, 219 aa, synthetic construct
DIVMTQSPLSLPVTPGEPVSISCRSSKSLLYSNGNTYLYWFLQKPGQSPQLLIYRMSNLASGVPDRFSGSGSGTAFTLKISRVEAEDVGVYYCMQHLEYPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 24 - protein, 10 aa, synthetic construct
GFSFNTYAMN

SEQ ID NO: 25 - protein, 10 aa, synthetic construct
GFTFNTYYMN

SEQ ID NO: 26 - protein, 10 aa, synthetic construct
GFEFNTYAMN

SEQ ID NO: 27 - protein, 10 aa, synthetic construct
GFEFNTYYMN

SEQ ID NO: 28 - protein, 19 aa, synthetic construct
RIRSKSNNYATYYADSVKD

SEQ ID NO: 29 - protein, 9 aa, synthetic construct
QVTGLAMDY

SEQ ID NO: 30 - protein, 16 aa, synthetic construct
RSSKSLLYSNGNTYLY

SEQ ID NO: 31 - protein, 7 aa, synthetic construct
RMSNLAS

SEQ ID NO: 32 - protein, 9 aa, synthetic construct
MQHLEYPLT

SEQ ID NO: 33 - protein, 450 aa, synthetic construct
EVQLVESGGGLVQPGGSLRLSCAASGFSFNTYAMNWVRQAPGKGLEWVARIRSKSNNYATYYADSVKDRFTISRDDSKSSLYLQMNSLKTEDTAVYYCVRQVTGLAMDYWGQGTTVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 34 - protein, 219 aa, synthetic construct
DIVMTQSPLSLPVTPGEPVSISCRSSKSLLYSNGNTYLYWFLQKPGQSPQLLIYRMSNLASGVPDRFSGSGSGTAFTLKISRVEAEDVGVYYCMQHLEYPLTFGQGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 35 - protein, 115 aa, synthetic construct
QVQLQQSGPELVKPGASVRIYCKASGYTFTTDYLHWVKKRPGQGLEWIGWIYPGKLITNYNEKFKDKATLTADKSSSTAYMHLSSLTSEDSAVYFCVRDPWFADWGQGTLVTVSA

SEQ ID NO: 36 - protein, 110 aa, synthetic construct
QAVVTQESALTTSPGETVTLTCRSSTGAVTPNNYANWVQEKPDHLFTGLIGGTNNRTPGVPARFSGSLIGDKAALTITGAQTEDEAIYFCALWYSNHFWVFGGGTKLTVL

SEQ ID NO: 37 - protein, 445 aa, synthetic construct
QVQLQQSGPELVKPGASVRIYCKASGYTFTTDYLHWVKKRPGQGLEWIGWIYPGKLITNYNEKFKDKATLTADKSSSTAYMHLSSLTSEDSAVYFCVRDPWFADWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 38 - protein, 216 aa, synthetic construct
QAVVTQESALTTSPGETVTLTCRSSTGAVTPNNYANWVQEKPDHLFTGLIGGTNNRTPGVPARFSGSLIGDKAALTITGAQTEDEAIYFCALWYSNHFWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 39 - protein, 8 aa, synthetic construct
GYTFTTDY

SEQ ID NO: 40 - protein, 8 aa, synthetic construct
IYPGKLIT

SEQ ID NO: 41 - protein, 8 aa, synthetic construct
VRDPWFAD

SEQ ID NO: 42 - protein, 9 aa, synthetic construct
TGAVTPNNY

SEQ ID NO: 43 - skipped

SEQ ID NO: 44 - protein, 10 aa, synthetic construct
ALWYSNHFWV

SEQ ID NO: 45 - protein, 10 aa, synthetic construct
GYTFTTDYLH

SEQ ID NO: 46 - protein, 17 aa, synthetic construct
WIYPGKLITNYNEKFKD

SEQ ID NO: 47 - protein, 6 aa, synthetic construct
DPWFAD

SEQ ID NO: 48 - protein, 14 aa, synthetic construct
RSSTGAVTPNNYAN

SEQ ID NO: 49 - protein, 7 aa, synthetic construct
GTNNRTP

SEQ ID NO: 50 - protein, 10 aa, synthetic construct
ALWYSNHFWV

SEQ ID NO: 51 - protein, 115 aa, synthetic construct
QVQLVQSGAEVKKPGSSVKISCKASGYTFTTDYLHWVRQAPGQGLEWIGWIYPGKLITNYNEKFKDRATLTADKSTSTAYMELSSLRSEDTAVYYCVRDPWFADWGQGTLVTVSS

SEQ ID NO: 52 - protein, 110 aa, synthetic construct
QAVVTQEPSLTVSPGGTVTLTCRSSTGAVTPNNYANWVQQKPGQAFRGLIGGTNNRAPWTPARFSGSLLGGKAALTLSGAQPEDEAEYYCALWYSNHFWVFGGGTKLTVL

SEQ ID NO: 53 - protein, 110 aa, synthetic construct
QAVVTQEPSLTVSPGGTVTLTCRSSTGAVTPNNYANWVQQKPGQAPRGLIGGTNNRAPWTPARFSGSLLGGKAALTLSGAQPEDEAEYYCALWYSNHFWVFGGGTKLTVL

SEQ ID NO: 54 - protein, 445 aa, synthetic construct
QVQLQQSGPELVKPGASVRIYCKASGYTFTTDYLHWVKKRPGQGLEWIGWIYPGKLITNYNEKFKDKATLTADKSSSTAYMHLSSLTSEDSAVYFCVRDPWFADWGQGTLVTVSAAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 55 - protein, 216 aa, synthetic construct
QAVVTQESALTTSPGETVTLTCRSSTGAVTPNNYANWVQEKPDHLFTGLIGGTNNRAPGVPARFSGSLIGDKAALTITGAQTEDEAIYFCALWYSNHFWVFGGGTKLTVLGQPKSSPSVTLFPPSSEELETNKATLVCTITDFYPGVVTVDWKVDGTPVTQGMETTQPSKQSNNKYMASSYLTLTARAWERHSSYSCQVTHEGHTVEKSLSRADCS

SEQ ID NO: 56 - protein, 7 aa, synthetic construct
GTNNRAP

SEQ ID NO: 57 - protein, 445 aa, synthetic construct
QVQLVQSGAEVKKPGSSVKISCKASGYTFTTDYLHWVRQAPGQGLEWIGWIYPGKLITNYNEKFKDRATLTADKSTSTAYMELSSLRSEDTAVYYCVRDPWFADWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 58 - protein, 217 aa, synthetic construct
QAVVTQEPSLTVSPGGTVTLTCRSSTGAVTPNNYANWVQQKPGQAFRGLIGGTNNRAPWTPARFSGSLLGGKAALTLSGAQPEDEAEYYCALWYSNHFWVFGGGTKLTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 59 - protein, 217 aa, synthetic construct
QAVVTQEPSLTVSPGGTVTLTCRSSTGAVTPNNYANWVQQKPGQAPRGLIGGTNNRAPWTPARFSGSLLGGKAALTLSGAQPEDEAEYYCALWYSNHFWVFGGGTKLTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC